(19) United States
(12) Patent Application Publication
(10) Pub. No.: US 2010/0279301 A1
(43) Pub. Date: Nov. 4, 2010

(54) METHODS AND COMPOSITIONS FOR DIAGNOSING BLADDER CANCER

(75) Inventors: Arul Chinnaiyan, Plymouth, MI (US); Rou Wang, Ann Arbor, MI (US); David Morris, Gallatin, TN (US); Scott Tomlins, Ann Arbor, MI (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/772,429

(22) Filed: May 3, 2010

Related U.S. Application Data
(60) Provisional application No. 61/175,177, filed on May 4, 2009.

(51) Int. Cl. C12Q 1/68

(52) U.S. Cl. 435/6

(57) ABSTRACT

The present invention relates to compositions and methods for cancer diagnosis, research and therapy, including but not limited to, cancer markers. In particular, the present invention relates to genes and gene panels as diagnostic markers and clinical targets for bladder cancer. Methods, compositions, and kits for diagnosing, determining, and determining risk of tumor aggressiveness are disclosed.

### CROSS REFERENCE TO RELATED APPLICATIONS

This application claims priority to U.S. Provisional Patent Application Ser. No. 61/175,177, filed: May 4, 2009, which is hereby incorporated by reference in its entirety.

### STATEMENT REGARDING FEDERALLY SPONSORED RESEARCH OR DEVELOPMENT

This invention was made with government support under grant number U01 CA113913 awarded by the National Institutes of Health. The government has certain rights in the invention.

### FIELD OF THE INVENTION

The present invention relates to compositions and methods for cancer diagnosis, research and therapy, including but not limited to, cancer markers. In particular, the present invention relates to genes and gene panels as diagnostic markers and clinical targets for bladder cancer.

### BACKGROUND OF THE INVENTION

The American Cancer Society estimates that in 2008 there were about 68,810 new cases of bladder cancer diagnosed in the United States (about 51,230 men and 17,580 women). Bladder cancer is the fourth most common cancer in men. The chance of a man developing this cancer at any time during his lifetime is about 1 in 27 and for a woman, 1 in 85. There were approximately 14,100 deaths due to bladder cancer in the United States in 2008. Risk factors for bladder cancer include smoking, race, age, gender, genetics, chronic bladder inflammation, prior chemotherapy or radiation therapy, exposure to arsenic, low fluid consumption, and workplace or environmental exposure to certain chemicals including aromatic amines, such as benzidine and beta-naphthylamine, which are sometimes used in the dye industry.

In developed countries, nearly all (˜97%) cases of bladder cancer involve transitional cell carcinoma (TCC). In this type of bladder cancer, cancerous cells within the urothelium (the inner lining of the bladder, also referred to as transitional epithelium) may be either papillate—having finger-like projections into the bladder interior—or flat. Papillary tumors are less likely to invade the underlying lamina propria and muscularis propia layers, while flat carcinomas are more likely to be invasive and aggressive. Other types of bladder cancer include squamous cell carcinoma, which is associated with the parasitic worm Schistosoma hematobium and which is therefore the most common type of bladder cancer in developing nations; adenocarcinoma of the bladder; and small cell carcinoma of the bladder.

Limited methods exist for diagnosing bladder cancer and staging tumors. Classical clinical diagnostic methods include observation of visible blood in the urine (hematuria); however, low levels of blood are not visible and must be detected through laboratory testing. Patients at risk for or suspected to have bladder cancer also may be examined by cytoscopy, and laboratory diagnostics may include urine cytology and culture, both of which are relatively time-consuming and low-throughput. In recent years, some urine biomarkers such as NMP22 and telomerase have been assessed, but generally found to have inadequate diagnostic value (Kapila et al. (2008) Cytopathology 19:369-374; Alvarez et al (2007) Curr. Opin. Urol. 17:341-346).

Staging bladder tumors is of particular concern, as determining the most appropriate treatment strategy requires knowing whether the tumor(s) have invaded, or are likely to invade, the underlying muscular layers. Biopsies are examined histologically, and patients may be monitored by imaging techniques including intravenous urography, retrograde pyelography, conventional X-ray radiography, CT scan, MRI, ultrasound, bone scan, and PET scan. Despite available methods, diagnosis and staging remains challenging.

There is a need for improved methods of detecting and staging bladder cancer tumors. The present invention addresses this need.

### SUMMARY OF THE INVENTION

The present invention relates to compositions and methods for cancer diagnosis, research and therapy, including but not limited to, cancer markers. In particular, the present invention relates to genes and gene panels as diagnostic markers and clinical targets for bladder cancer.

In some embodiments, the invention provides a method for analyzing a bladder tumor, comprising: quantifying the level of expression in a sample (e.g., a bladder tissue sample) of at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 60-70, 70-80, 80-90, 90 or more) gene such as ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53); and determining the risk of the bladder tumor to invade bladder muscular tissue of the subject based on the level expression of at least one of the aforementioned genes. In some embodiments, over-expression of at least one gene such as ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), and UBE2C (ubiquitin-conjugating enzyme E2C) is indicative of increased risk of said bladder tumor invading muscular tissue of the bladder. In some embodiments, under-expression of at least one gene such as ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53) is indicative of increased risk of said bladder tumor invading muscular tissue of the bladder.

In some embodiments, the stage of the tumor is known (e.g., has been determined by histological analysis). In some embodiments, the stage of the tumor has been determined to be stage Ta, T is, or T1. In some embodiments, the stage of the tumor has been determined to be stage T2 or higher. In some embodiments, the stage of the tumor is not known. In some embodiments, the bladder tissue sample comprises a tumor biopsy sample. In some embodiments, the method further comprises comparing expression levels of genes tested in the first sample to the expression levels of the same genes in a second sample. In some embodiments, this second sample comprises a benign bladder tissue sample from the same subject. In some embodiments, the second sample comprises a benign bladder tissue sample from a different subject. In some embodiments, the second sample comprises a historical bladder tissue sample from the same subject. In some embodiments, the expression levels of genes tested in the first sample are compared to numerical cutoff values. In some embodiments, the numerical cutoff values are related to average values occurring in a subject or subjects not having bladder cancer. In some embodiments, the method is executed more than once. In some embodiments, the method is executed at periodic intervals. The intervals may be more often than once a week, every 1 week, every 2 weeks, every 3 weeks, every 4 weeks, every 4-8 weeks, every 8-12 weeks, every 3-4 months, every 4-5 months, every 5-6 months, every 6-7 months, every 7-8 months, every 8-9 months, every 9-10 months, every 10-11 months, every 11-12 months, every 1-2 years, every 2-3 years, every 3-5 years, every 5-10 years, or a period that is greater than every 10 years.

In some embodiments, the present invention provides a method for diagnosing bladder cancer, said method comprising: quantifying the level of expression in a bladder tissue sample of at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 60-70, 70-80, 80-90, 90 or more) gene such as ACTN1 (actinin, alpha 1), ADAM12 (ADAM metallopeptidase domain 12), APOCl (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53); and determining the presence of at least one bladder tumor based on the level of expression of at least one of the aforementioned genes. In some embodiments, over-expression of at least one gene such as ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), and UBE2C (ubiquitin-conjugating enzyme E2C) is indicative of presence of at least one bladder tumor. In some embodiments, under-expression of at least one gene such as ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53) is indicative of presence of at least one bladder tumor. In some embodiments, the bladder tissue sample comprises a tumor biopsy. In some embodiments, the method for diagnosing bladder cancer further comprises the step of comparing expression levels of the aforementioned genes to the expression level of the same genes in a second sample. In some embodiments, the second sample comprises a benign bladder tissue sample from the same subject. In some embodiments, the second sample comprises a historical bladder tissue sample from the same subject. In some embodiments, the method for diagnosing bladder cancer is executed more than once at periodic intervals. The intervals may be more often than once a week, every 1 week, every 2 weeks, every 3 weeks, every 4 weeks, every 4-8 weeks, every 8-12 weeks, every 3-4 months, every 4-5 months, every 5-6 months, every 6-7 months, every 7-8 months, every 8-9 months, every 9-10 months, every 10-11 months, every 11-12 months, every 1-2 years, every 2-3 years, every 3-5 years, every 5-10 years, or a period that is greater than every 10 years. In some embodiments, the method for diagnosing bladder cancer finds use in determining the stage of at least one bladder tumor.

In some embodiments, the present invention provides a kit comprising reagents configured to quantify the expression of one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 60-70, 70-80, 80-90, 90 or more) or more genes such as ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53). In some embodiments, the kit comprises a QPCR card.

In some embodiments, the present invention provides a method for diagnosing bladder cancer, comprising: quantifying the level of expression of at least one gene such as ACTN1 (actinin, alpha 1) and CDC25C (cell division cycle 25 homolog C), and determining the presence of at least one bladder tumor based on the level of expression of at least one of the aforementioned genes. In some embodiments, the present invention provides a method for analyzing a bladder tumor, comprising: quantifying the level of expression of at least one gene such as ACTN1 (actinin, alpha 1) and CDC25C (cell division cycle 25 homolog C), and determining the risk of the bladder tumor to invade bladder muscular tissue of a subject based on the level of expression of at least one of the aforementioned genes.

In some embodiments, the present invention comprises a kit including at least one (e.g., 1, 2, 3, 4, 5, 6, 7, 8, 9, 10 or more) reagent useful, necessary or sufficient to practice any of the methods of the present invention. Kit embodiments of the present invention may comprise, e.g., primers, probes, antibodies, buffers, detection agents, positive controls, negative controls, software, instructions, microarrays, and/or QPCR cards.

Additional embodiments of the invention are described herein.

### DESCRIPTION OF THE FIGURES

FIG. 1 shows the development of a bladder cancer progression signature based on comparative meta-profiling. A, Nine bladder cancer and three multi-cancer microarray datasets were uploaded into Oncomine for bioinformatics analysis. B, Detailed list of 96 genes whose assays were loaded onto the TLDA QPCR card.

FIG. 2 shows freedom from progression to T2 disease in 96 patients with non muscle-invasive bladder cancer based on a 57 gene signature. A-D, Kaplan-Meier estimates of freedom from T2 progression for the 57-gene signature in all non-T2 patients (A), T1 patients only (C), and Ta patients only (D). Patients were divided into high- and low-risk groups based on their cross-validated predictions of risk to T2 progression, using the median risk score as a cutoff. Kaplan-Meier estimates of stage at resection as a predictor of T2 progression are illustrated in (B) for comparison. E, Multivariable analysis of gender, pathological stage at resection, and gene signature score in relation to likelihood of progression to T2 disease.

FIG. 3 shows a heatmap representation of the 57 gene signature for bladder cancer progression.

FIG. 4 shows immunohistochemical staining of two metasignature candidates, ACTN1 and CDC25B. Representative immunostaining of ACTN1 (A1-A3) and CDC25B (A4-A6) across bladder cancer progression in non-invasive bladder cancer (A1, A4), invasive bladder cancer (A2, A5) and metastatic deposits in lymph nodes (A3, A6). The higher-magnification insets represent various expression levels of cytoplasmic staining for ACTN1 and nuclear staining for CDC25B. Boxplots of median product scores for ACTN1 (B) and CDC25B (C) by evaluation status show a clear trend of increasing product score by cancer progression.

FIG. 5 shows gene expression reproducibility across batches.

FIG. 6 shows molecular concept maps of signature genes. Network view of molecular concepts map analysis of over-expressed (A) and under-expressed (B) genes (black nodes) in the final 57-gene signature.

### DEFINITIONS

To facilitate an understanding of the present invention, a number of terms and phrases are defined below:

As used herein, the term “gene upregulated in cancer” refers to a gene that is expressed (e.g., mRNA or protein expression) at a higher level in cancer (e.g., bladder cancer, bladder tumor) relative to the level in other tissues. In some embodiments, genes upregulated in cancer are expressed at a level at least 10%, preferably at least 25%, even more preferably at least 50%, still more preferably at least 100%, yet more preferably at least 200%, and most preferably at least 300% higher than the level of expression in other tissues.

As used herein, the term “gene downregulated in cancer” refers to a gene that is expressed (e.g., mRNA or protein expression) at a lower level in cancer (e.g., bladder cancer, bladder tumor) relative to the level in other tissues. In some embodiments, genes down-regulated in cancer are expressed at a level at least 10%, preferably at least 25%, even more preferably at least 50%, still more preferably at least 100%, yet more preferably at least 200%, and most preferably at least 300% lower than the level of expression in other tissues.

As used herein, the term “gene upregulated in bladder tissue” refers to a gene that is expressed (e.g., mRNA or protein expression) at a higher level in bladder tissue relative to the level in other tissue. In some embodiments, genes upregulated in bladder tissue are expressed at a level at least 10%, preferably at least 25%, even more preferably at least 50%, still more preferably at least 100%, yet more preferably at least 200%, and most preferably at least 300% higher than the level of expression in other tissues. In some embodiments, genes upregulated in bladder tissue are exclusively expressed in bladder tissue.

As used herein, the terms “detect”, “detecting” or “detection” may describe either the general act of discovering or discerning or the specific observation of a detectably labeled composition.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “amplification oligonucleotide” refers to an oligonucleotide that hybridizes to a target nucleic acid, or its complement, and participates in a nucleic acid amplification reaction. An example of an amplification oligonucleotide is a “primer” that hybridizes to a template nucleic acid and contains a 3′ OH end that is extended by a polymerase in an amplification process. Another example of an amplification oligonucleotide is an oligonucleotide that is not extended by a polymerase (e.g., because it has a 3′ blocked end) but participates in or facilitates amplification. Amplification oligonucleotides may optionally include modified nucleotides or analogs, or additional nucleotides that participate in an amplification reaction but are not complementary to or contained in the target nucleic acid. Amplification oligonucleotides may contain a sequence that is not complementary to the target or template sequence. For example, the 5′ region of a primer may include a promoter sequence that is non-complementary to the target nucleic acid (referred to as a “promoter-primer”). Those skilled in the art will understand that an amplification oligonucleotide that functions as a primer may be modified to include a 5′ promoter sequence, and thus function as a promoter-primer. Similarly, a promoter-primer may be modified by removal of, or synthesis without, a promoter sequence and still function as a primer. A 3′ blocked amplification oligonucleotide may provide a promoter sequence and serve as a template for polymerization (referred to as a “promoter-provider”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “QPCR” means quantitative polymerase chain reaction, wherein the products of the reaction are quantified by any means. Methods for quantification of PCR products are known in the art and include but are not limited to the inclusion of a fluorescent dye(s) in the PCR reaction, such that the fluorescent signal may be monitored throughout the amplification, preferably in real time and without need for separation of reaction products. Signal may be imparted through the use of nonspecific dyes that bind any double-stranded product regardless of sequence (including but not limited to Syber Green dye); sequence-specific methods, such as TaqMan probes; nucleotide-specific methods, such as inclusion of one or more labeled nucleotides in the reaction; and non-fluorometric methods e.g. relying on detection of chromogenic, radioisotopic, or otherwise labeled products; bead-based flow cytometric assays (e.g., Luminex); or quantitative mass spectrometric methods allowing direct analysis of amplified product. The latter methods may be conducted on aliquots of the reaction in real time, or may be conducted for the purpose of endpoint analysis. By quantifying signal associated with PCR product, one skilled in the art can compare how many rounds (cycles) of amplification are necessary to reach a detectable signal which is indicatory of the initial amount of template in the sample. This signal level (threshold level) is always set in the exponential phase of amplification where samples can be directly compared. Traditionally, a target gene of interest has been compared to some kind of reference gene, to take into account differences in extraction yields, reverse transcription efficiencies and differences caused by inhibitors within the samples. One skilled in the art appreciates the inherent need to identify suitable reference genes with stable expression throughout the study intended. In many cases housekeeping genes such as GAPDH, β-tubulin and β-actin have been used; however such genes may not be suitable for all purposes, as the expression of many are also regulated in certain situations and can therefore cause erroneous results.

As used herein, a “gene expression pattern” or “gene expression profile” or “gene signature” refers to the relative expression of a set of genes correlated with the classification of samples for cancer and particularly bladder cancer, as well as the expression of genes correlated with the type and stage of bladder tumor. Moreover, the terms “gene expression pattern” or “gene expression profile” or “gene signature” indicate that combined pattern of the results of the analysis of the level of expression of two or more biomarkers of the invention including any or all of the biomarkers of the invention. A gene expression pattern or gene expression profile or gene signature can result from the measurement of expression of the RNA and/or the protein expressed by the gene corresponding to the biomarkers of the invention. In the case of RNA it refers to the RNA transcripts transcribed from genes corresponding to the biomarker of the invention. In the case of protein it refers to proteins translated from the genes corresponding to the biomarker of the invention. For example, techniques to measure expression of the RNA products of the biomarkers of the invention includes, PCR based methods (including RT-PCR and QPCR) and non PCR based methods as well as microarray analysis. To measure protein products of the biomarkers of the invention, techniques include western blotting and ELISA analysis. In some embodiments, at least one member of the gene set analyzed is characteristically up-regulated in bladder tumor cells, such that increased level of expression of such gene(s) in a sample finds use in diagnosing or determining the risk of bladder cancer. In some embodiments, at least one member of the gene set analyzed is characteristically down-regulated in bladder tumor cells, such that decreased level of expression of such gene(s) in a sample finds use in diagnosing or determining the risk of bladder cancer. One skilled in the art will appreciate that adding or removing members of a set to be analyzed for gene expression pattern, gene expression profile, or gene signature may improve the diagnostic or risk predictive power from a clinical standpoint. Therefore, 1, 1-2, 2-5, 5-10, 10-20, or 20 or more members may be added or removed from a gene set or protein biomarker set used to analyze a gene expression pattern, gene expression profile, or gene signature.

### DETAILED DESCRIPTION OF THE INVENTION

Approximately 75% of newly-diagnosed patients with bladder cancers will have disease confined to the urothelium or lamina propria (stages Ta, T is, and T1). These non-muscle invasive tumors account for significant morbidity given recurrence rates of 50-70% (NCCN clinical practice guidelines in oncology: bladder cancer (including upper tract tumors and urothelial carcinoma of the prostate): National Comphensive Cancer Network, 2008) and need for cystoscopic surveillance with re-treatment. Furthermore, 10-15% of these tumors will progress to muscle invasion or higher (T2-4) (Millan-Rodriguez et al (2000) J. Urol., 163:73-78), with significantly worsened prognosis and 5-year overall survival rates of 50-60% (Shariat et al (2008) Cancer, 112:315-325). To date, there has been no reliable means of predicting tumor progression other than clinical judgment, published risk estimates for tumor stage and grade, or burgeoning clinical nomograms (Millan-Rodriguez et al. (2000) J. Urol., 163:73-78; Shariat et al. (2005) J. Urol. 173:1518-1525).

In parallel to these ongoing clinical questions in bladder cancer, there has been significant development of DNA microarray gene expression analysis over the last decade. Microarray analysis has become a high-throughput method of measuring the cancer transcriptome and can distinguish cancer from normal tissues, identify cancer subtypes, and predict recurrence or treatment response. Some cancers, such as breast cancer, have been studied extensively with microarray analysis, generating gene signatures to guide clinical management (Paik et al. (2004) New Engl. J. Med. 351:2817-2826; van't Veer et al. (2002) Nature 415:530-536). Other cancers, such as bladder cancer, have been investigated infrequently with microarray analysis. A recent query of the Affymetrix publication database and PubMed confirms the disparity in microarray research attention between bladder and breast cancer: bladder cancer is linked to 38 Affymetrix and 93 PubMed publications, while breast cancer is linked to 254 Affymetrix and 935 PubMed citations.

Furthermore, clinical application of microarray gene signatures has been difficult historically given the lack of reproducibility with independent validation. Small cohorts and variable microarray platforms may explain the minimal overlap seen between gene signatures.

Preferably, a gene signature that will be used for risk stratification is well-validated across various, independent patient populations. Previously, the limitations of varied platforms and analyses were sought to be overcome through comparative meta-profiling of microarray datasets to characterize a common transcriptional profile across most cancer types (Rhodes et al. (2004) PNAS 101:9309-9314; herein incorporated by reference in its entirety). Comparative meta-profiling generates gene signatures from the overlap of independent microarray datasets, limiting the noise of spuriously identified genes and accentuating true underlying signature patterns. Furthermore quantitative polymerase-chain reaction (QPCR), relative to microarrays, is more reproducible, possesses a larger dynamic range, and is a clinically more tractable platform for diagnostics and prognostics development.

In experiments conducted during the development of embodiments of the present invention, comparative meta-profiling was used to analyze published bladder cancer microarray datasets and determine common transcriptional profiles associated with cancer development, recurrence, progression, and outcome. By combining these platforms, populations, and analyses, it was possible to tailor the large number of genes in microarray studies to a smaller and more robust metasignature of 96 genes associated with aggressive behavior in bladder cancer. These 96 genes were pre-configured onto a clinically applicable, high-throughput QPCR card. Gene expression values were quantified for 96 frozen tumor tissue specimens. Ultimately, 57 genes were selected which significantly differentiated between non-muscle-invasive and muscle-invasive tumors. This study assessed the ability of a 57-gene signature to predict probability of progression of non-muscle-invasive bladder tumors to T2 disease. This signature aids in the identification of non-muscle-invasive bladder cancers that are more likely to progress and for which earlier definitive therapy such as cystectomy may be offered. More generally speaking, a systematic approach is presented to take advantage of publicly available microarray gene expression datasets on tumors and convert them into a more clinically applicable platform.

### I. Cancer Markers

Experiments conducted during the course of development of embodiments of the present invention identified genes described herein as being overexpressed or underexpressed in bladder cancer. The present invention thus provides DNA, RNA and protein based diagnostic methods that either directly or indirectly detect overexpression or underexpression of the genes described herein (see, e.g., Table 7). Some embodiment of the present invention also provide compositions and kits for diagnostic purposes.

The diagnostic methods of the present invention may be qualitative or quantitative. Quantitative diagnostic methods may be used, for example, to discriminate between indolent and aggressive cancers via a cutoff or threshold level. Where applicable, qualitative or quantitative diagnostic methods may also include amplification of target, signal or intermediary (e.g., a universal primer).

A. Sample

Any patient sample suspected of containing overexpression or underexpression of, for example, genes listed in Table 7 is tested according to the methods of the present invention. By way of non-limiting examples, the sample may be tissue (e.g., a bladder biopsy sample or post-surgical tissue), blood, urine, or a fraction thereof (e.g., plasma, serum, urine supernatant, urine cell pellet or breast cells). In preferred embodiments, the sample is a tissue sample obtained from a biopsy (e.g., needle biopsy, aspiration biopsy or surgically obtained biopsy) or following surgery (e.g., bladder biopsy).

In some embodiments, the patient sample undergoes preliminary processing designed to isolate or enrich the sample for transcripts or polypeptides corresponding to genes listed in Table 7 or cells that contain transcripts or polypeptides corresponding to genes listed in Table 7. A variety of techniques known to those of ordinary skill in the art may be used for this purpose, including but not limited: centrifugation; immunocapture; cell lysis; and, nucleic acid target capture (See, e.g., EP Pat. No. 1 409 727, herein incorporated by reference in its entirety).

B. DNA and RNA Detection

In some embodiments, overexpression or underexpression of genes listed in Table 7 is detected as mRNA or genomic DNA (e.g., copy number decrease or increase) using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety) is the first commercialized single-molecule sequencing platform. This method does not require clonal amplification. Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run. Other emerging single molecule sequencing methods real-time sequencing by synthesis using a VisiGen platform (Voelkerding et al., Clinical Chem., 55: 641-658, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition. Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503) utilizes reaction wells 50-100 nm in diameter and encompassing a reaction volume of approximately 20 zeptoliters (10×10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

2.1 FISH

In some embodiments, sequences are detected using fluorescence in situ hybridization (FISH). The preferred FISH assays for the present invention utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

Specific protocols for performing FISH are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

2.2 Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Genomic DNA and mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified nucleic acids corresponding to the genes listed in Table 7 can be detected by any conventional means. For example, in some embodiments, such nucleic acids are detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

C. Protein Detection

In some embodiments, the present invention provides methods of detecting protein and levels of protein encoded by any one or more of the genes listed in Table 7. Proteins are detected using a variety of protein techniques known to those of ordinary skill in the art, including but not limited to: mass spectrometry, protein sequencing, and immunoassays.

1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

2. Mass Spectrometry

In some embodiments, mass spectrometry is used to identify proteins. The present invention is not limited by the nature of the mass spectrometry technique utilized for such analysis. For example, techniques that find use with the present invention include, but are not limited to, ion trap mass spectrometry, ion trap/time-of-flight mass spectrometry, time of flight/time of flight mass spectrometry, quadrupole and triple quadrupole mass spectrometry, Fourier Transform (ICR) mass spectrometry, and magnetic sector mass spectrometry. The following description of mass spectroscopic analysis is illustrated with ESI oa TOF mass spectrometry. Those skilled in the art will appreciate the applicability of other mass spectroscopic techniques to such methods.

In some embodiments, proteins are analyzed simultaneously to determine molecular weight and identity. A fraction of the effluent is used to determine molecular weight by either MALDI-TOF-MS or ESI oa TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI oa TOF. The molecular weight is matched to the appropriate digest fingerprint by correlating the molecular weight total ion chromatograms (TICs) with the UV-chromatograms and by calculation of the various delay times involved. The resulting molecular weight and digest mass fingerprint data can then be used to search for the protein identity via web-based programs like MSFit (UCSF).

3. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies that are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

D. Data Analysis

In some embodiments, a computer-based analysis program is used to translate the raw data generated by the detection assay (e.g., the presence, absence, or amount of expression of at least one of the genes listed in Table 7) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. In Vivo Imaging

In some further embodiments, expression of genes listed in Table 7 is detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence or level of expression of genes listed in Table 7 in an animal (e.g., a human or non-human mammal). For example, in some embodiments, mRNA or protein correlated to the genes listed in Table 7 is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express at least one of the genes listed in Table 7 at a decreased level relative to the level in non-cancerous tissues (e.g., prostate cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for at least one of the proteins encoded by the genes listed in Table 7 are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

F. Compositions & Kits

Compositions for use in the diagnostic methods of the present invention include, but are not limited to, probes, amplification oligonucleotides, and antibodies. Particularly preferred compositions detect the level of expression of at least one of the genes listed in Table 7 in a sample.

Any of these compositions, alone or in combination with other compositions of the present invention, may be provided in the form of a kit. For example, the single labeled probe and pair of amplification oligonucleotides may be provided in a kit for the amplification and detection of at least one of the genes listed in Table 7. The kit may include any and all components necessary or sufficient for assays including, but not limited to, the reagents themselves, buffers, control reagents (e.g., tissue samples, positive and negative control sample, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered.

The probe and antibody compositions of the present invention may also be provided in the form of an array.

### II. Therapeutic Methods

In some embodiments, the present invention provides methods of customizing cancer (e.g., prostate cancer) therapy. For example, in some embodiments, the expression of at least one of the genes listed in Table 7 in a sample from a patient is assayed. Patients with altered expression of one of the genes listed in Table 7 are then treated with an therapy that restores the level of the gene or genes to that found in patients without disease or in the same patient prior to onset of disease (e.g., reduction therapy or replacement therapy). The customized treatment methods of the present invention provide the advantage of therapy directed to a specific target at the molecular level. The use of unnecessary treatments that are not effective can be avoided.

The present invention is not limited to a particular therapy. Exemplary therapies are described below.

### A. Small Molecule Therapies

In some preferred embodiments, small molecular therapeutics are utilized. In certain embodiments, small molecule therapeutics targeting regulators of genes listed in Table 7 are identified, for example, using the drug screening methods of the present invention.

### B. Antisense

In some embodiments, the methods involve, for example, the delivery of nucleic acid molecules targeting at least one gene listed in Table 7 or members of pathways in which these genes are involved within cancer cells (e.g., bladder). For example, in some embodiments, the present invention employs compositions comprising oligomeric antisense compounds, particularly oligonucleotides, for use in modulating the function of nucleic acid molecules encoding upstream modulators of at least one of the genes listed in Table 7, ultimately modulating the amount of expression of at least one of the genes listed in Table 7. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of upstream modulators of at least one of the genes listed in Table 7. In the context of the present invention, “modulation” means either an increase (stimulation) or a decrease (inhibition) in the expression of a gene. For example, expression may be inhibited to potentially prevent tumor growth, inhibition of complement mediated lysis, angiogenesis and proliferation associated with over- or underexpression of at least one of the genes listed in Table 7 (e.g., in bladder cancer).

C. shRNA

In some embodiments, the present invention provides shRNAs that inhibit the expression of at least one of the genes listed in Table 7 (e.g., in bladder cancer cells). A short hairpin RNA (shRNA) is a sequence of RNA that makes a tight hairpin turn that can be used to silence gene expression via RNA interference. shRNA typically uses a vector introduced into cells and utilizes a promoter (e.g., the U6 promoter) to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it.

D. siRNA

In some embodiments, the present invention provides siRNAs that inhibit the expression of at least one of the genes listed in Table 7 (e.g., in bladder cancer cells). siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (see, e.g., Brummelkamp et al, Science 2002; 296:550-3; and Holen et al, Nucleic Acids Res. 2002; 30:1757-66). An important factor in the design of siRNAs is the presence of accessible sites for siRNA binding. Bahoia et al., (J. Biol. Chem., 2003; 278: 15991-15997) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

### E. Delivery of Nucleic Acids

Introduction of molecules carrying genetic information into cells is achieved by any of various methods including, but not limited to, directed injection of naked DNA constructs, bombardment with gold particles loaded with the constructs, macromolecule mediated gene transfer using, for example, liposomes, biopolymers, and the like, and ex vivo transfection and/or gene therapy followed by transplantation. The present invention is not limited to a particular approach for introducing molecules carrying genetic information to a subject (e.g., a human subject, a non-human subject). In some embodiments, the methods employ a nanovector delivery system (e.g., a cationic liposome-mediated gene transfer system; a lipoplex) for delivering gene therapeutics to a subject. Current approaches to deliver gene therapeutics to cancer patients often employ either viral or non-viral vector systems. Viral vector-directed methods show high gene transfer efficiency but are deficient in several areas. The limitations of a viral approach are related to their lack of tumor targeting and to residual viral elements that can be immunogenic, cytopathic, or recombinogenic. To circumvent these problems, progress has been made toward developing non-viral, pharmaceutical formulations of gene therapeutics for in vivo human therapy, particularly nanovector delivery systems (e.g., cationic liposome-mediated gene transfer systems). Indeed, there are multiple clinical trials underway using nanovector delivery systems for gene delivery, and liposomes for delivery of chemotherapeutics such as doxorubicin are already on the market for breast cancer chemotherapy. Features of nanovector delivery systems (e.g., cationic liposomes) that make them versatile and attractive include: ease of preparation, ability to complex large pieces of DNA/RNA, the ability to transfect many different types of cells, including non-dividing cells, and the lack of immunogenicity or biohazard activity.

In some embodiments, the nanovector delivery systems (e.g., cationic liposomes) are configured to bear a ligand recognized by a cell surface receptor (e.g., to increase desired targeting to, for example, a tumor). The nanovector delivery systems are not limited to a particular ligand recognized by a cell surface receptor. In some embodiments, the ligand is recognized by a cell surface receptor specific to a tumor. In some embodiments, the ligand is transferrin (Tf). In some embodiments, the ligand is a single chain antibody fragment (scFv) (e.g., specific to Tf). Receptor-mediated endocytosis represents a highly efficient internalization pathway in eukaryotic cells. The presence of a ligand on a nanovector delivery systems (e.g., cationic liposome; lipoplex) facilitates the entry of DNA into cells. Recently, a tumorspecific, ligand-targeting, self-assembled nanoparticle-DNA lipoplex system designed for systemic gene therapy of cancer was developed (see, e.g., U.S. Pat. No. 6,749,863; Tibbetts R S, Genes Dev 2000; 14:2989-3002; Zou L, Science 2003; 300: 1542-1548; each of which is herein incorporated by reference). These nanovector systems employ transferrin (Tf) or a single chain antibody fragment (scFv) against the transferrin receptor which is overexpressed in the majority of human cancers, including pancreatic cancer (see, e.g., Busino L, et al., Nature 2003; 426: 87-91). TfR-scFv-targeted nanovectors were recently approved by the FDA for clinical testing and the first Phase I clinical trial for non-viral systemic p53 gene therapy is ongoing.

Some methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is incorporated herein by reference in their entireties.

### G. Antibody Therapy

In some embodiments, the present invention provides antibodies that target tumors that over- or underexpress at least one of the genes listed in Table 7. Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a polypeptide encoded by a gene listed in Table 7 or a regulator thereof, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

### H. Pharmaceutical Compositions

A therapeutic nucleic acid molecule of the present invention can be adapted for use to treat any disease, infection or condition associated with gene expression, and other indications that can respond to the level of gene product in a cell or tissue, alone or in combination with other therapies. For example, a therapeutic nucleic acid molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074), poly(lactic-co-glycolic)acid (PLGA) and PLCA microspheres (see for example U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. Many examples in the art describe CNS delivery methods of oligonucleotides by osmotic pump, (see Chun et al., 1998, Neuroscience Letters, 257, 135-138, D'Aldin et al., 1998, Mol. Brain. Research, 55, 151-164, Dryden et al., 1998, J. Endocrinol., 157, 169-175, Ghimikar et al., 1998, Neuroscience Letters, 247, 21-24) or direct infusion (Broaddus et al., 1997, Neurosurg. Focus, 3, article 4). Other routes of delivery include, but are not limited to oral (tablet or pill form) and/or intrathecal delivery (Gold, 1997, Neuroscience, 76, 1153-1158). More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein. The siNAs of the instant invention can be used as pharmaceutical agents. Pharmaceutical agents prevent, modulate the occurrence, or treat (alleviate a symptom to some extent, preferably all of the symptoms) of a disease state in a subject.

Thus, embodiments of the present invention feature a pharmaceutical composition comprising one or more nucleic acid(s) of the invention in an acceptable carrier, such as a stabilizer, buffer, and the like. The polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes siRNA molecules to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant a composition or formulation that allows for the effective distribution of nucleic acid molecules in the physical location most suitable for their desired activity. Non-limiting examples of agents suitable for formulation with the nucleic acid molecules of embodiments of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies include, but are not limited to, material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation based on their ability to avoid accumulation in metabolically aggressive MPS tissues such as the liver and spleen.

Embodiments of the present invention also include compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In some embodiments, the methods of the present invention directed toward increasing expression and/or activity of at least one of the genes listed in Table 7 or transcripts or polypeptides correlating with at least one of the genes listed in Table 7, further involve co-administration with an anti-cancer agent (e.g., chemotherapeutic). The present invention is not limited by type of anti-cancer agent co-administered. Indeed, a variety of anti-cancer agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-cancer agents include: Antiproliferative agents (e.g., Piritrexim Isothionate), Antiprostatic hypertrophy agent (e.g., Sitogluside), Benign prostatic hypertrophy therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Still other anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-Cl-2′ deoxyadenosine; Fludarabine-PO₄; mitoxantrone; mitozolomide; Pentostatin; and Tomudex.

One particularly preferred class of anticancer agents are taxanes (e.g., paclitaxel and docetaxel). Another important category of anticancer agent is annonaceous acetogenin. Other cancer therapies include hormonal manipulation. In some embodiments, the anti-cancer agent is tamoxifen or the aromatase inhibitor arimidex (i.e., anastrozole).

### III. Antibodies

Polypeptides encoded by at least one of the genes listed in Table 7 or regulator proteins thereof, including fragments, derivatives and analogs thereof, may be used as immunogens to produce antibodies having use in the diagnostic, research, and therapeutic methods described below. The antibodies may be polyclonal or monoclonal, chimeric, humanized, single chain or Fab fragments. Various procedures known to those of ordinary skill in the art may be used for the production and labeling of such antibodies and fragments. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975).

### V. Drug Screening Applications

In some embodiments, the present invention provides drug screening assays (e.g., to screen for anticancer drugs). The screening methods of the present invention utilize cancer markers identified using the methods of the present invention (e.g., at least one of the genes listed in Table 7). For example, in some embodiments, the present invention provides methods of screening for compounds that alter (e.g., increase or decrease) the expression of cancer marker genes or regulators of cancer marker genes. The compounds or agents may interfere with transcription, by interacting, for example, with the promoter region. The compounds or agents may interfere with mRNA produced from regulators of at least one of the genes listed in Table 7 (e.g., by RNA interference, antisense technologies, etc.). The compounds or agents may interfere with pathways that are upstream or downstream of the biological activity of transcripts or polypeptides correlated with at least one of the genes in Table 7. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present invention and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly prostate cancer.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### VII. Transgenic Animals

The present invention contemplates the generation of transgenic animals comprising an exogenous cancer marker gene (e.g., at least one of the genes listed in Table 7) of the present invention or mutants and variants thereof (e.g., truncations or single nucleotide polymorphisms). In other embodiments, the animals are knockout animals lacking functional copies of at least one of the genes listed in Table 7. In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

### Example 1

Methods described in Example 1 were employed in the course of experiments described in Example 2. Additional methods and results are described in Example 3.

### Sample Selection and Preparation

Cases with available frozen bladder cancer tissue from time of transurethral resection of the bladder tumor (TURBT) were selected from those patients enrolled in the bladder cancer database at the University of Michigan. All samples were collected with the informed consent of the patients and prior institutional review board approval. Samples were selected based on pathologic stage at time of TURBT (Ta, T1, or T2), presence of transitional cell carcinoma, lack of mixed or variant histology, and no previous intravesical or systemic therapy within one year of TURBT. Overall, 100 samples fit these criterion and clinical information was collected regarding initial tumor grade and stage at time of TURBT, recurrence, local or distant progression, and disease-specific and overall mortality. Patients were characterized into two groups: non-muscle-invasive (Ta, T1) cancers that did not demonstrate evidence of progression to T2 disease during follow-up, and any stage tumors that were pathologic T2 at TURBT or demonstrated progression to T2 disease, local or distant metastasis, or cancer-specific death during follow-up.

Each frozen tissue sample was sectioned into seven 20-micron sections, and RNA isolation was performed using Trizol extraction (Invitrogen, Carlsbad, Calif.). QPCR was performed using Taqman dye on the Applied Biosystems 7900HT Fast Real-Time PCR system.

Reproducibility across batches was investigated by performing repeat gene expression measurement of 16 tumor samples (see Example 3).

Additionally, twelve benign bladder frozen specimens were identified from adjacent benign tissue in radical cystectomy cases, as obtained from the frozen tissue bank and tissue procurement service at the University of Michigan, and RNA extraction was performed from dissected epithelium-rich areas. These samples were also run on the TLDA cards (see Example 3).

RNA yield quantification was performed with the Nanodrop 1000 spectrophotometer (Thermo Fisher Scientific, Waltham, Mass.).

### QPCR Analysis

Gene expression was normalized relative to the average of five housekeeping genes (ACTB (beta-actin), CYCS (Cytochrome C), GAPDH (Glyceraldehyde-3-phosphate Dehydrogenase), and SDHA (Succinate Dehydrogenase Complex, subunit A)); the values were then log 2-transformed. 18S (18S rRNA gene) was excluded from this housekeeping average as raw Ct (threshold cycle) values consistently ran in the 2-3 cycle range. Samples were excluded from analysis if they demonstrated weak QPCR signal (raw Ct value for GAPDH >28, or housekeeping average >31).

### Molecular Concepts Map Analysis

Molecular concepts map (MCM) analysis computes pairwise associations between gene sets to create an ‘enrichment network’ of associations across all available signatures, arising from a variety of cancer types, pathways, mechanisms and drugs (Tomlins et al (2007) Nat. Genet. 39:41-51; herein incorporated by reference in its entirety). This compendium of over 14,000 ‘molecular concepts,’ or sets of biologically connected genes. A gene set of interest can then be investigated for its functional overlap with other gene sets and integration with biologic concepts (see Example 3).

### Tissue Microarray Construction and Immunohistochemical Evaluation

Two genes, actinin (ACTN1) and cell division cycle 25B (CDC25B), were identified from the metasignature; these had commercially available antibodies and were chosen for immunohistochemistry (IHC) analysis utilizing bladder cancer progression tissue microarray (TMA). Immunohistochemistry was performed on the tissue microarray using mouse monoclonal antibodies against CDC25B (Labvision 1188-p1; 1 in 50 dilution) and ACTN1 (Santa Cruz sc-17829; 1 in 50 dilution) and standard avidin-biotin complex techniques, as described previously (Mehra et al (2005) Cancer Res. 65:11259-11264) (see Example 3).

### Statistical Analysis

To obtain the gene signature, univariate Wilcoxon rank-sum tests were first used to identify genes significantly differentiating T2 from non-T2 tumors, with a two-sided p-value <0.05 considered statistically significant. These expression values were log-transformed using the transformation log(expression+1). Gene expression raw Ct values that were missing initially had log-values imputed as zero, implying there was no expression of that gene relative to housekeeping genes. To reduce the influence of outliers, the distribution of each gene's expression values was truncated at the third upward standard deviation. Principal components analysis was used to reduce this set of genes into a smaller number of variables explaining >75% of the data variance, and the principal components were used as predictors in a multivariate Cox regression model for T2 progression. Patients who had already progressed to T2 at TURBT were coded as having time-to-event=0. The best Cox model was chosen using a backwards selection algorithm incorporating the Akaike Information Criterion (AIC) for model comparison.

In order to evaluate the signature's predictive power, leave-one-out cross-validation was performed, resulting in a predicted probability of progression to T2 for each Ta and T1 patient at TURBT. These cross-validated predictions were used to stratify non-T2 patients into high- and low-risk groups for T2 progression, using the median predicted probability as the cutoff. Differences in outcome were evaluated using Kaplan-Meier curves and log-rank tests. Additionally, AIC was used to select a best multivariate Cox regression model for progression to T2 using known clinical variables as predictors, and the likelihood ratio test was used to evaluate the significance of the signature when added to this clinical model. Associations between clinical variables and T2 progression were assessed using univariate Cox models and likelihood ratio tests.

For immunohistochemistry analysis, one-way ANOVA was used to compare distributions of the median product scores by group. F-tests were used to compare competing models, and comparisons between groups were made using Tukey's Honest Significant Difference (HSD) procedure (for pairwise comparisons) and Scheffe's method (other comparisons).

All statistical analyses were performed using R, version 2.7.0.

### Example 2

### Comparative Meta-Profiling of Existing Microarray Data

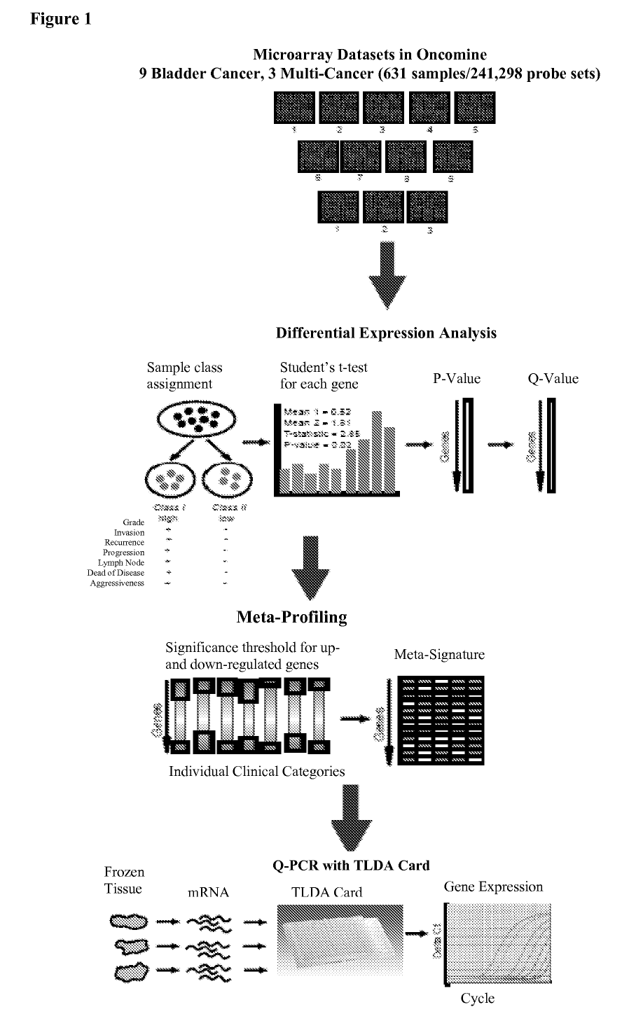

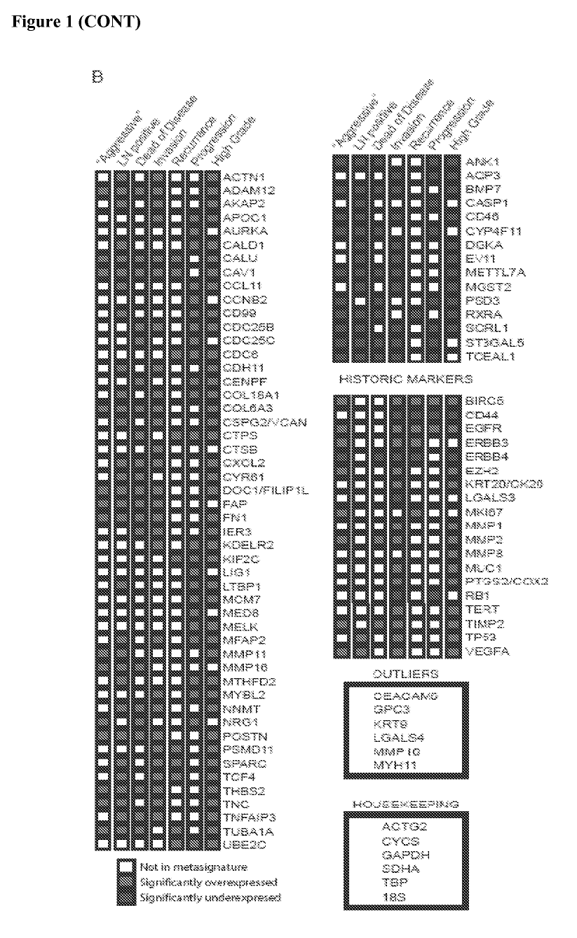

Nine previously published bladder cancer microarray profiling datasets and three multi-cancer microarray profiling datasets (Table 1) were identified. These publicly available microarray data sets were uploaded into Oncomine (Rhodes et al (2007) Neoplasia 9:166-180; herein incorporated by reference in its entirety), an online compendium and advanced analysis platform for gene expression datasets. The flow diagram of comparative meta-profiling leading to the creation of a TLDA card is detailed in FIG. 1A.

TABLE 1
Clinical characteristics of the patient cohort.

| | Eligible Patients (n = 96) |
|---|---|
| Age (yrs) | |
| Mean ± SD | 69 ± 11 |
| (range) | (41, 92) |
| Gender (M, F) | 75, 21 |
| Race | |
| Caucasian | 90 |
| African-American | 4 |
| Other | 2 |
| Pathologic Stage/Grade | |
| Ta[HG, LG] | 31[8, 23] (32.3%) |
| T1[HG, LG] | 31[26, 5] (32.3%) |
| T2 | 34 (35.4%) |
| Associated CIS | 23 (24%) |
| Developed Metastases | 23 (24%) |
| Death | 38 (39.6%) |
| Median Follow-up for | 2.4 |
| non-T2 Patients (years) | |

For each of the microarray profiling studies, the clinical information of all profiled samples was reviewed, including cancer grade and stage, recurrence, local or distant progression, and patient death. Ultimately, six clinical categories were defined: cancer grade, muscle invasion, recurrence, progression to higher stage, positive lymph node status, and death from disease. A seventh clinical category for overall aggressiveness was devised, combining progression, positive lymph nodes, or death from disease. Individual samples were assigned to classes for each analysis, and in each study, genes were assessed in Oncomine for differential expression between these classes with Student's t-test, to create meta-profiles for each clinical category. Ultimately, genes were included in the bladder cancer meta-signature if they were significantly over-expressed in at least three clinical category meta-profiles or under-expressed in at least two (FIG. 1B); this led to the inclusion of 50 over-expressed and 15 under-expressed genes. Six outlier genes in the datasets were also identified by Oncomine analysis and included in the metasignature, as well as six housekeeping genes and 19 historic markers. Cumulatively, this resulted in a meta-signature of 96 genes of interest (Table 2).

TABLE 2
Clinical category meta-profiles created
with comparative meta-profiling.

| | | | | Study | |
|---|---|---|---|---|---|
| Clinical | | Size | Number | inclusion | |
| State | Expression | of list | of genes | cutoff | p-value |
| HG | Up | Small | 90 | 6 of 8 | 0.05 |
| | | Large | 205 | 5 of 8 | 0.05 |
| | Down | Small | 28 | 6 of 8 | 0.05 |
| | | Large | 145 | 5 of 8 | 0.05 |
| Progression | Up | Small | 46 | 3 of 4 | 0.05 |
| | | Large | 152 | 2 of 4 | 0.02 |
| | Down | Small | 44 | 3 of 4 | 0.05 |
| | | Large | 206 | 2 of 4 | 0.03 |
| Invasion | Up | Small | 34 | 5 of 7 | 0.005 |
| | | Large | 194 | 4 of 7 | 0.008 |
| | Down | Small | 66 | 5 of 7 | 0.01 |
| | | Large | 153 | 4 of 7 | 0.005 |
| Recurrence | Up | Small | 23 | 3 of 5 | 0.05 |
| | | Large | 71 | 3 of 5 | 0.05 |
| | Down | Small | 8 | 3 of 5 | 0.05 |
| LN+ | Up | Small | 44 | 3 of 3 | 0.02 |
| | | Large | 220 | 2 of 3 | 0.01 |
| | Down | Small | 32 | 3 of 3 | 0.05 |
| | | Large | 228 | 2 of 3 | 0.02 |
| DOD | Up | Small | 24 | 3 of 3 | 0.05 |
| | | Large | 195 | 2 of 3 | 0.04 |
| | Down | Small | 22 | 3 of 3 | 0.05 |
| | | Large | 201 | 2 of 3 | 0.03 |
| Poor Outcome | Up | Small | 6 | 6 of 8 | 0.05 |
| | | Large | 39 | 5 of 8 | 0.05 |
| | Down | Small | 6 | 6 of 8 | 0.05 |
| | | Large | 33 | 5 of 8 | 0.05 |

Number of genes included was determined by applying the minimum number of studies with significance above the p-value cutoff. HG, histological grade; LN+, lymph node positive; DOD, dead due to disease.

These 96 genes were then preloaded onto a 96A-well format Taqman Low Density Array TLDA) card (Applied Biosystems, Inc., Foster City, Calif.), which allows for multiplex high-throughput QPCR measurements. Five batches of ten cards each were constructed.

### Characteristics of Patients Used for Development of Gene Signatures

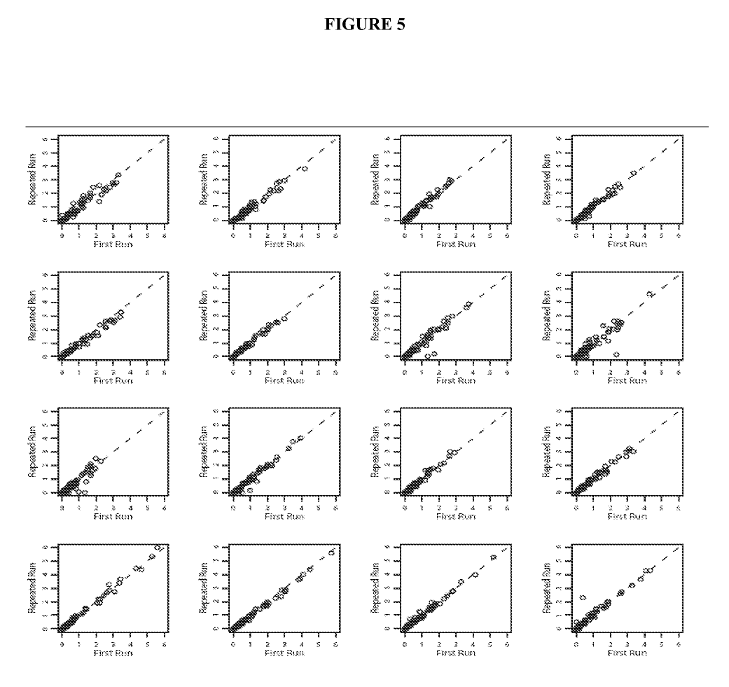

Frozen tumor sections were available for all 100 patients initially selected from the tissue bank and 12 benign bladder specimens (total n=112). One benign and four tumor samples were eliminated from final analysis, secondary to low expression of housekeeping genes. The final cohort consisted of 107 samples: 96 tumor samples, with a distribution of 42 non-progressing tumors and 54 progressing or T2 tumors, and 11 benign bladder samples. There was high QPCR reproducibility across batches (FIG. 5). Patient demographics for the final 96 tumor samples are listed in Table 1. Median follow-up in non-T2 patients for whom predictions were being made was 2.4 years.

Univariate analysis revealed that pathologic stage (T1 vs. Ta) was the only significant clinical predictor of T2 progression (p=0.01). Histological grade (high vs. low) approached significance as a predictor of T2 progression (p=0.08), but a within-stage analysis revealed that grade was not predictive of progression for Ta or T1 patients (Table 3).

TABLE 3
List of 96 genes and assay IDs pre-loaded onto the Taqman Low Density Array card;
in the case that the assay ID maps to more than one RefSeq ID, the first is listed.

| | | | |
|---|---|---|---|
| | | | Applied Biosystems, |
| Gene Symbol | Full Name | RefSeq ID | Inc. Assay Number |

Housekeeping

| | | | |
|---|---|---|---|
| ACTG2 | Actin, beta | NM_001101.3 | Hs00242273_m1 |
| CYCS | Cytochrome-C, somatic | NM_018947.4 | Hs01588974_g1 |
| GAPDH | glyceraldehyde-3-phosphate dehydrogenase | NM_002046.3 | Hs99999905_m1 |
| SDHA | succinate dehydrogenase complex, subunit A, | NM_004168.2 | Hs00188166_m1 |
| | flavoprotein (Fp) | | |
| TBP | TATA box binding protein | NM_003194.3 | Hs00427620_m1 |
| 18S (excluded) | 18S ribosomal RNA | NR_003286.1 | Hs99999901_s1 |

Up-Regulated

| | | | |
|---|---|---|---|
| ACTN1 | Actinin, alpha 1 | NM_001102.2 | Hs00241650_m1 |
| ADAM12 | ADAM metallopeptidase domain 12 | NM_003474.3 | Hs01106104_m1 |
| AKAP2 | A kinase (PRKA) anchor protein 2 | NM_001004065.3 | Hs00200512_m1 |
| APOC1 | apolipoprotein C-I | NM_001645.3 | Hs03037377_m1 |
| AURKA | aurora kinase A | NM_003600.2 | Hs00269212_m1 |
| CALD1 | caldesmon 1 | NM_004342.5 | Hs00189021_m1 |
| CALU | calumenin | NM_001219.2 | Hs00154230_m1 |
| CAV1 | caveolin 1 | NM_001753.3 | Hs00184697_m1 |
| CCL11 | chemokine (C-C motif) ligand 11 | NM_002986.2 | Hs00237013_m1 |
| CCNB2 | cyclin B2 | NM_004701.2 | Hs00270424_m1 |
| CD99 | CD99 molecule | NM_002414.3 | Hs00365982_m1 |
| CDC25B | cell division cycle 25 homolog B | NM_004358.3 | Hs00244740_m1 |
| CDC25C | cell division cycle 25 homolog C | NM_001790.3 | Hs00156407_m1 |
| CDC6 | cell division cycle 6 homolog | NM_001254.3 | Hs00154374_m1 |
| CDH11 | cadherin 11, type 2 | NM_001797.2 | Hs00156438_m1 |
| CENPF | centromere protein F, 350/400ka | NM_016343.3 | Hs00193201_m1 |
| COL18A1 | collagen, type XVIII, alpha 1 | NM_030582.3 | Hs00181017_m1 |
| COL6A3 | collagen, type VI, alpha 3 | NM_004369.2 | Hs00365098_m1 |
| CSPG2/VCAN | versican | NM_004385.2 | Hs00171642_m1 |
| CTPS | CTP synthase | NM_001905.2 | Hs00157163_m1 |
| CTSB | cathepsin B | NM_001908.3 | Hs00157194_m1 |
| CXCL2 | chemokine (C-X-C motif) ligand 2 | NM_002089.3 | Hs00236966_m1 |
| CYR61 | cysteine-rich, angiogenic inducer, 61 | NM_001554.3 | Hs00155479_m1 |
| DOC1/FILIP1L | filamin A interacting protein 1-like | NM_001042459.1 | Hs00706279_s1 |
| FAP | fibroblast activation protein, alpha | NM_004460.2 | Hs00189476_m1 |
| FN1 | fibronectin 1 | NM_002026.2 | Hs00365052_m1 |
| IER3 | immediate early response 3 | NM_003897.3 | Hs00174674_m1 |
| KDELR2 | KDEL (Lys-Asp-Glu-Leu) endoplasmic | NM_006854.3 | Hs00199277_m1 |
| | reticulum protein retention receptor 2 | | |
| KIF2C | kinesin family member 2C | NM_006845.3 | Hs00199232_m1 |
| LIG1 | ligase I, DNA, ATP-dependent | NM_000234.1 | Hs00172073_m1 |
| LTBP1 | latent transforming growth factor beta binding | NM_000627.2 | Hs00386448_m1 |
| | protein 1 | | |
| MCM7 | minichromosome maintenance complex | NM_005916.3 | Hs00428518_m1 |
| | component 7 | | |
| MED8 | mediator complex subunit 8 | NM_052877.3 | Hs00364620_m1 |
| MELK | maternal embryonic leucine zipper kinase | NM_014791.2 | Hs00207681_m1 |
| MFAP2 | microfibrillar-associated protein 2 | NM_017459.1 | Hs00250063_m1 |
| MMP11 | matrix metallopeptidase 11 | NM_005940.3 | Hs00171829_m1 |
| MMP16 | matrix metallopeptidase 16 | NM_005941.4 | Hs01095537_m1 |
| MTHFD2 | methylenetetrahydrofolate dehydrogenase | NM_001040409.1 | Hs00759197_s1 |
| | (NADP+ dependent) 2, methenyltetrahydrofolate | | |
| | cyclohydrolase | | |
| MYBL2 | v-myb myeloblastosis viral oncogene homolog | NM_002466.2 | Hs00231158_m1 |
| | (avian)-like 2 | | |
| NNMT | nicotinamide N-methyltransferase | NM_006169.2 | Hs00196287_m1 |
| NRG1 | neuregulin 1 | NM_013960.2 | Hs00247652_m1 |
| POSTN | periostin, osteoblast specific factor | NM_006475.1 | Hs00170815_m1 |
| PSMD11 | proteasome (prosome, macropain) 26S subunit, | NM_002815.2 | Hs00160660_m1 |
| | non-ATPase, 11 | | |
| SPARC | secreted protein, acidic, cysteine-rich | NM_003118.2 | Hs00234160_m1 |
| TCF4 | transcription factor 4 | NM_001083962.1 | Hs00162613_m1 |
| THBS2 | thrombospondin 2 | NM_003247.2 | Hs00170248_m1 |
| TNC | tenascin C | NM_002160.2 | Hs00233648_m1 |
| TNFAIP3 | tumor necrosis factor, alpha-induced protein 3 | NM_006290.2 | Hs00234712_m1 |
| TUBA1A | tubulin, alpha 1a | NM_006009.2 | Hs00362387_m1 |
| UBE2C | ubiquitin-conjugating enzyme E2C | NM_181799.1 | Hs00738962_m1 |

Down-Regulated

| | | | |
|---|---|---|---|
| ANK1 | ankyrin 1, erythrocytic | NM_000037.3 | Hs00252830_m1 |
| AQP3 | aquaporin 3 | NM_004925.3 | Hs00185020_m1 |
| BMP7 | bone morphogenetic protein 7 | NM_001719.1 | Hs00233477_m1 |
| CASP1 | caspase 1, apoptosis-related cysteine peptidase | NM_001223.3 | Hs00354832_m1 |
| CD46 | CD46 molecule, complement regulatory protein | NM_002389.3 | Hs00174507_m1 |
| CYP4F11 | cytochrome P450, family 4, subfamily F, | NM_021187.2 | Hs00430476_m1 |
| | polypeptide 11 | | |
| DGKA | diacylglycerol kinase, alpha 80 kDa | NM_001345.4 | Hs00176278_m1 |
| EVI1 | ecotropic viral integration site 1 | NM_005241.2 | Hs01118675_m1 |
| METTL7A | methyltransferase like 7A | NM_014033.3 | Hs00204042_m1 |
| MGST2 | microsomal glutathione S-transferase 2 | n/a | Hs00182064_m1 |
| PSD3 | pleckstrin and Sec7 domain containing 3 | NM_015301.3 | Hs00535354_s1 |
| RXRA | retinoid X receptor, alpha | NM_002957.3 | Hs00172565_m1 |
| SORL1 | sortilin-related receptor, L(DLR class) A | NM_003105.4 | Hs00268342_m1 |
| | repeats-containing | | |
| ST3GAL5 | ST3 beta-galactoside alpha-2,3-sialyltransferase 5 | NM_001042437.1 | Hs00187405_m1 |
| TCEAL1 | transcription elongation factor A (SII)-like 1 | NM_001006639.1 | Hs00231846_m1 |

Historic

| | | | |
|---|---|---|---|
| BIRC5 | baculoviral IAP repeat-containing 5 | NM_001012270.1 | Hs00153353_m1 |
| CD44 | CD44 molecule | NM_000610.3 | Hs00153310_m1 |
| EGFR | epidermal growth factor receptor | NM_005228.3 | Hs00193306_m1 |
| ERBB3 | v-erb-b2 erythroblastic leukemia viral oncogene | NM_001005915.1 | Hs00176538_m1 |
| | homolog 3 | | |
| ERBB4 | v-erb-a erythroblastic leukemia viral oncogene | NM_001042599.1 | Hs00171783_m1 |
| | homolog 4 | | |
| EZH2 | enhancer of zeste homolog 2 | NM_004456.3 | Hs00544830_m1 |
| KRT20 | keratin 20 | NM_019010.1 | Hs00300643_m1 |
| LGALS3 | lectin, galactoside-binding, soluble, 3 | NM_002306.2 | Hs00173587_m1 |
| MKI67 | antigen identified by monoclonal antibody Ki-67 | NM_002417.3 | Hs00267195_m1 |
| MMP1 | matrix metallopeptidase 1 | NM_002421.2 | Hs00899653_g1 |
| MMP2 | matrix metallopeptidase 2 | NM_004530.2 | Hs00234422_m1 |
| MMP8 | matrix metallopeptidase 8 | NM_002424.1 | Hs01029057_m1 |
| MUC1 | mucin 1, cell surface associated | NM_001018016.1 | Hs00159357_m1 |
| PTGS2 | prostaglandin-endoperoxide synthase 2 | NM_000963.1 | Hs00153133_m1 |
| RB1 | retinoblastoma 1 | NM_000321.2 | Hs01078066_m1 |
| TERT | telomerase reverse transcriptase | NM_198253.2 | Hs00162669_m1 |
| TIMP2 | TIMP metallopeptidase inhibitor 2 | NM_003255.4 | Hs00234278_m1 |
| TP53 | tumor protein p53 | NM_000546.3 | Hs00153340_m1 |
| VEGFA | vascular endothelial growth factor A | NM_001025366.1 | Hs00173626_m1 |

Outlier

| | | | |
|---|---|---|---|
| CEACAM5 | carcinoembryonic antigen-related cell adhesion | NM_004363.2 | Hs00237075_m1 |
| | molecule 5 | | |
| GPC3 | glypican 3 | NM_004484.2 | Hs00170471_m1 |
| KRT9 | keratin 9 | NM_000226.2 | Hs00413861_m1 |
| LGALS4 | lectin, galactose binding, soluble 4 | n/a | Hs00196223_m1 |
| MMP10 | matrix metallopeptidase 10 | NM_002425.1 | Hs00233987_m1 |
| MYH11 | myosin, heavy chain 11, smooth muscle | NM_001040113.1 | Hs00224610_m1 |

### 57-Gene Signature

The 107 bladder tissue samples described above were then run on the pre-configured 96-element TLDA cards. Fifty-seven genes demonstrated significantly differential expression between T2 and non-T2 tumors (p<0.05), with an estimated false discovery rate of 1.1%. This set consisted of 37 over-expressed and 20 under-expressed genes in T2 tumors. Of the 37 over-expressed genes, 32 (86.5%) were predicted to be over-expressed in aggressive cancers from meta-profiling and the remainder were historic markers; 12/20 (60%) under-expressed genes were predicted to be under-expressed from meta-profiling, with seven historic markers or outliers and one gene, MMP16, initially predicted to be over-expressed. Further gene signature details are described in Example 3.

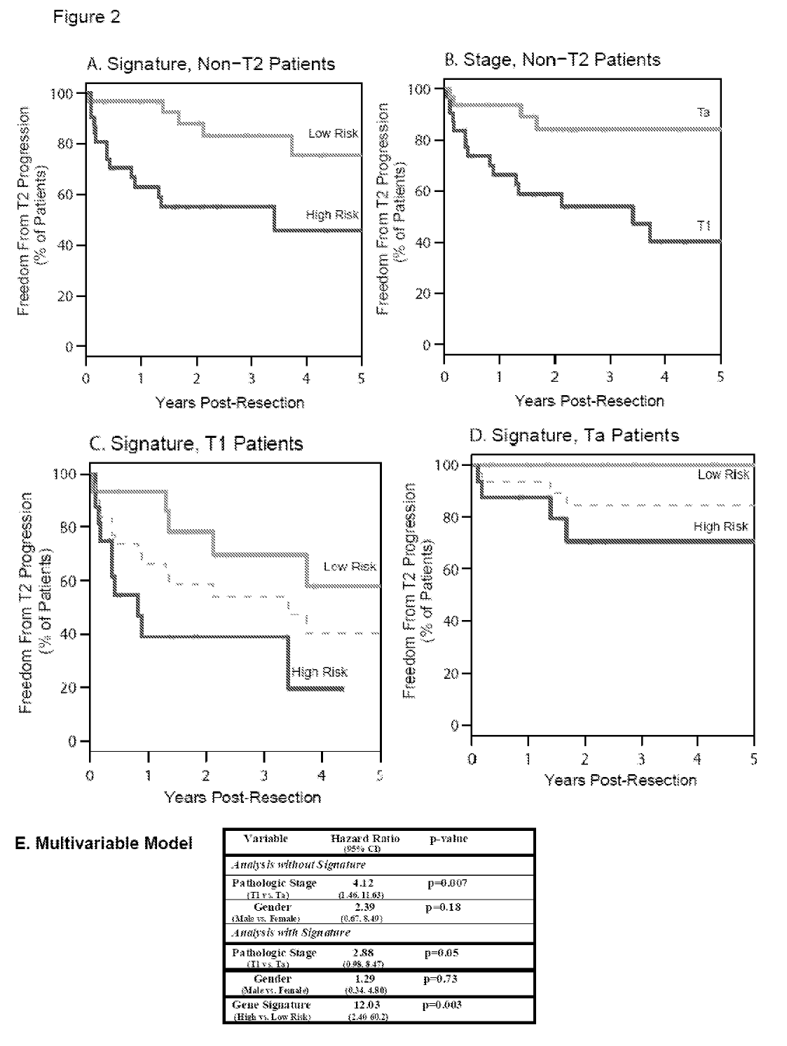

Whether this gene signature was associated with progression of Ta and T1 tumors to muscle-invasive disease was investigated. Outcomes for the Ta and T1 tumors during the first five years after resection are demonstrated in FIG. 2. Using the 57-gene signature to divide this population into high- and low-risk groups, high-risk patients exhibited a higher rate of progression to T2 disease within two years [(45% vs. 12%, p=0.003) (FIG. 2A)]. As expected, stage alone was a significant predictor of T2 progression, with more T1 patients experiencing T2 progression than Ta patients (FIG. 2B, p=0.007). Importantly, however, the gene signature prediction maintained significance within T1 tumors [(61% vs. 22%, p=0.02), FIG. 2C)] and Ta tumors [(29% vs. 0%, p=0.03), FIG. 2D], demonstrating that this gene signature provides additional risk stratification beyond stage alone. This difference in outcomes is most pronounced for T1 patients in the first year of follow-up, during which 7% of predicted low-risk T1 patients progressed to T2 disease, versus 61% of predicted high-risk T1 patients.

In a multivariate Cox model utilizing clinical parameters, the best model retained only stage and gender as significant predictors of progression to T2 disease (FIG. 2E). The gene signature, however, provided significant ability to predict progression, independent of stage and gender (likelihood ratio test: p=0.002).

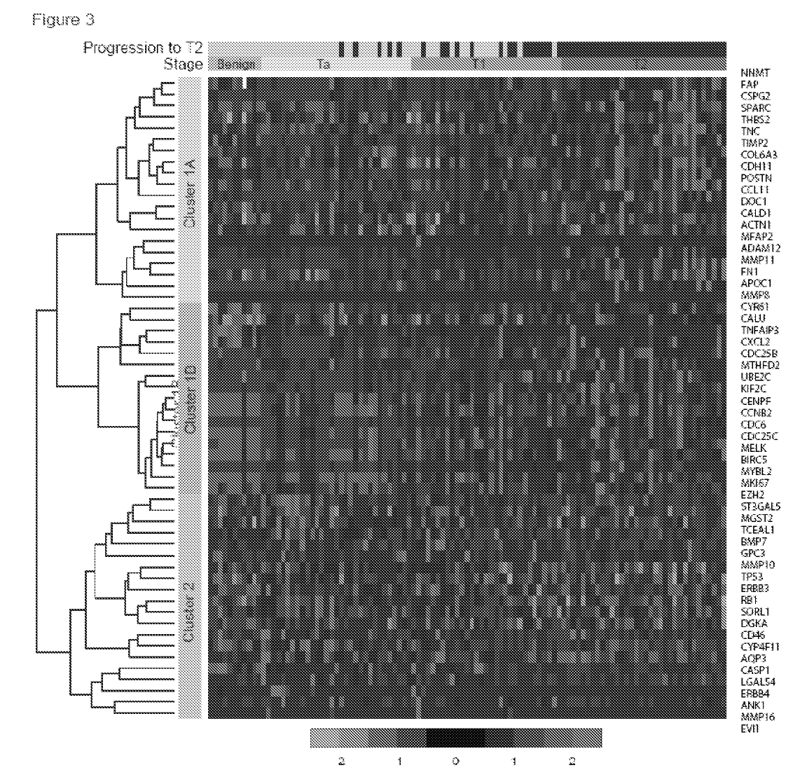

A heat map comparing the 57 individual genes of the signature and all available samples in the cohort is shown in FIG. 3. Hierarchical clustering of genes demonstrated the 37 over-expressed (Cluster 1) and 20 under-expressed genes (Cluster 2) in T2 disease. In the over-expressed gene set, two smaller gene subsets can be appreciated from hierarchical clustering (Clusters 1A and 1B). The 20 under-expressed genes in T2 disease are also relatively over-expressed in Ta patients without progression. The benign samples demonstrate relative under-expression of Cluster 1B and Cluster 2 genes. Cluster 2 genes are up-regulated with progression from benign to Ta disease, but down-regulated again in transition to T2 disease.

In an attempt to integrate the selected genes into a functional framework, the over-expressed and under-expressed gene sets were investigated in the context of MCM analysis, an established bioinformatics tool that was developed during experiments conducted during the development of the present invention. This concepts-based analysis of the 57 gene signature demonstrated enrichment of cell adhesion and extracellular matrix invasion pathways as well as cell cycle regulation and mitosis in the up-regulated genes, confirming the importance of these programs in progression to muscle invasion in bladder cancer (Sanchez-Carbayo et al (2002) Cancer Res. 62:6973-6980). For the under-expressed genes, these demonstrated overlap with under-expressed genes in poorly differentiated lung and invasive breast carcinomas; also, included in the list of down-regulated genes are several well-known tumor suppressors, including p53 and RB1 (retinoblastoma 1). See Example 3 for further details.

### Immunohistochemical Analysis

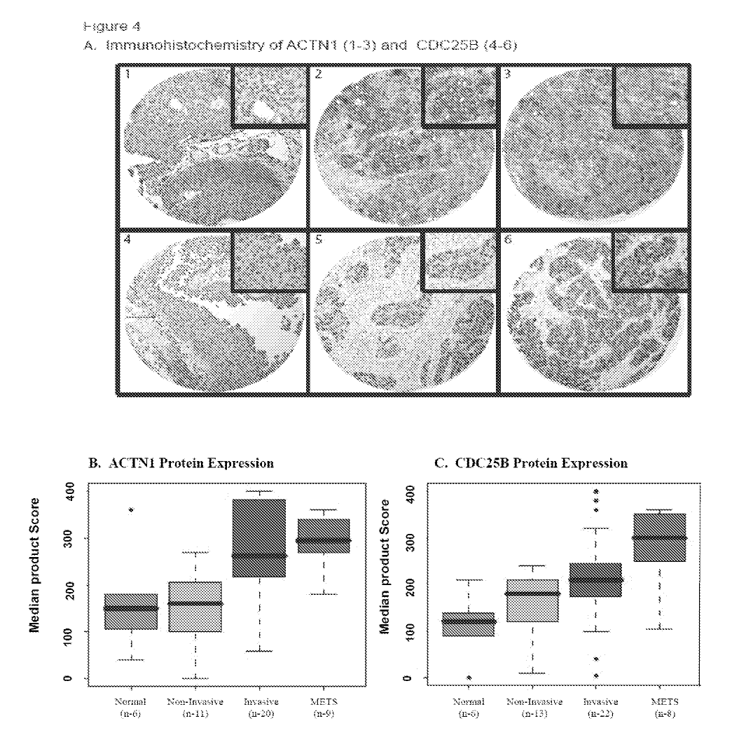

To further validate the components of the 57-gene signature using orthogonal approaches, members of the signature were identified for which immunohistochemistry-compatible antibodies were available to assess protein expression. Antibodies to ACTN1 and CDC25B were identified and Immunohistochemistry was performed to investigate protein expression in situ on a bladder cancer progression tissue microarray (FIG. 4). Both markers demonstrated homogenous staining of the tissue cores, with predominantly cytoplasmic expression for ACTN1 and nuclear expression for CDC25B. ACTN1 showed the most significant individual group comparison difference between the non-invasive and either invasive or metastatic groups (Tukey's HSD: p=0.003 for each) (FIG. 4B). CDC25B expression demonstrated a more linear trend with disease severity (p=0.0002) (FIG. 4C). More detailed information is available in Example 3.

The accurate designation of the 10-30% of non-muscle-invasive bladder cancers which will progress to muscle invasion has yet to be perfected, and currently relies upon pathologic staging and grading and continued surveillance with the gold standards of cystoscopy and urine cytology. Urine cytology, however, lacks sensitivity for low grade tumors (Lotan et al. (2003) Urology 61:109-118; Wiener et al. (1993) Acta Cytol. 37:163-169), and cystoscopic detection may occur months after the development of muscle-invasive disease, depending on the surveillance interval. Earlier detection of progression to muscle-invasive disease provides a survival benefit given the significant decrease in long-term survival of patients with muscle-invasive disease (due to the presence of concomitant micrometastasis). Additionally, patients with non-muscle-invasive cancers who progress to T2 on surveillance demonstrate similarly poor patterns of survival after cystectomy as patients presenting with T2 disease (Lee et al. (2007) Urology 69:1068-1072). Furthermore, while there exist multiple urine-based tests to detect incipient or recurrent bladder cancer (Lotan et al. (2003) Urology 61:109-118; O'Donnell (2007) Semin. Oncol. 34:85-97), there are no widely-used modalities to risk-stratify patients beyond initial cancer detection. Comprehensive nomograms estimate risks of recurrence and progression, but have not gained wide acceptance in the United States; these often require clinical information not always readily available, such as tumor multiplicity and size (Sylvester et al. (2006) 49:466-475), or incorporate single bladder tumor markers (Shariat et al. (2005) J. Urol. 173:1518-1525). A tumor-specific multi-gene signature may provide a more comprehensive picture of potential tumor aggressiveness.

Microarray gene-expression profiling provides a tremendous amount of information, but is difficult to translate into a clinical prognostic tool given the large number of genes involved (Ramaswamy (2004) N Engl. J. Med. 350:1814-1816) and required time and expertise. QPCR is more clinically applicable, especially when working with a small group of highly-selected genes of interest. Methods used during the development of embodiments of the present invention are applicable across many cancer types—namely, compiling publicly available microarray data for bioinformatics analysis, generating a larger list of robust genes involved in aggressive behavior, and deriving a smaller QPCR gene signature in order to predict an outcome of interest. In this study, the most essential transcripts are summarized from available bladder cancer microarray data into a select group of 96 genes, and further tailored to a prognostic gene set of 57 genes with the use of a pre-loaded, high-throughput QPCR card. This resulted in a clinically feasible test, utilizing a small amount of frozen bladder tumor available from the time of TURBT, to provide a tumor-specific gene signature that helps predict progression in non-muscle invasive cancers.

Specifically, patients who were designated as high-risk by the gene signature were more likely to have demonstrated progression to T2 disease than those who were designated as low-risk; this predictive ability surpassed information provided by pathologic stage alone, as the differences in rates of progression remained significant within stage. This provided evidence that a gene signature can provide additional risk stratification beyond pathologic information alone, particularly in light of the fact that inter-observer variability exists in tumor staging and grading, both in bladder (Bol et al. (2003) J. Urol. 169:1291-1294; Murphey et al. (2002) J. Urol. 168:968-972) and other cancers (Robbins et al. (1995) Hum. Pathol. 26:873-879). Given the use of electrocautery during TURBT, it can also be difficult to assess margin status accurately, and a re-staging TURBT is standard for pathologic T1 disease to assess for missed muscle invasion (Schwaibold et al. (2006) BJU Int. 97:1199-1201). Specifically, this gene signature possessed excellent predictive ability in the T1 tumor cohort during the first year following TURBT and more than half of patients predicted to be high-risk had progressed to T2 disease or higher. For the Ta cohort, patients with progression were classified appropriately as high-risk. Having this type of tumor-specific genetic information available at the time of TURBT allows for more tailored risk stratification in combination with other clinical parameters, such as age and surgical fitness, and adverse pathologic features, such as perineural or lymphovascular invasion and variant histology (for example, squamous or micropapillary differentiation). A more expedited offering of cystectomy as an alternative to intravesical bacillus Calmette-Guerin (BCG) therapy and surveillance, or following initial BCG failure is offered to T1 patients with high-risk clinicopathologic features who are also predicted to be at high risk for progression by the gene signature; in this dataset, more than 60% of high-risk T1 patients demonstrated progression in the first year. For Ta patients with a high-risk gene score, some of these patients may not progress fully to T2 disease, but the clinician's threshold for progression may be lowered such that alternative treatments are discussed soon after initial intravesical therapy failure.

Two genes from the signature whose protein expression has not been described explicitly in bladder cancer were chosen for immunohistochemistry analysis. ACTN1 has been shown to possess different splicing patterns in T2 versus Ta tumors (Thorsen et al. (2008) Mol. Cell. Proteomics 7:1214-1224), indicating an ability to utilize ACTN1 in stage separation; and in fact, ACTN1 protein expression was significantly different between nonmuscle-invasive and invasive or metastatic bladder cancers. CDC25B has been shown to be up-regulated in progressing bladder tumors in a previous microarray study (Dyrskjot et al. (2005) 11:4029-4036), correlating with its gradually up-regulated protein expression on immunohistochemistry. More information about these genes is available in Example 3.

TIMP2 has been studied in the context of bladder cancer metastases to the lung, with up-regulated expression concordant to tumor stage (Nicholson et al. (2004) 64:7813-7821). There has also been extensive work performed on the cooperative interactions of alterations of tumor suppressors p53 and RB1 in bladder cancer development and progression (Cordon-Cardo et al. (1997) 57:1217-1221; Markl et al. (1998) Cancer Res. 58:5348-5353). Furthermore, previous studies have demonstrated that overall cell cycle dysregulation is necessary for uroepithelial transformation and cell adhesion dysregulation is commonly found in tumor progression of uroepithelial neoplasms (Sanchez-Carbayo et al. (2002) Cancer Res. 62:6973-6980)).

### Example 3

### Methods

### Comparative Meta-Profiling

Nine previously published bladder cancer microarray profiling datasets and three multi-cancer microarray profiling datasets (Table 4) were uploaded into Oncomine, representing 631 samples, measuring between 1,168 and 59,619 genes. Values from all data sets were log-transformed and median-centered.

TABLE 4
List of 9 bladder cancer and 3 multi-cancer microarray studies used for
comparative meta-profiling to generate a bladder cancer meta-signature.

| Oncomine | Lead | | GEO | | No. |
|---|---|---|---|---|---|
| Study Name | Author | PMID | Accession # | N | Genes |
| Bladder Cancer Studies | | | | | |
| Blaveri_Bladder_3 | Blaveri E | 15930339 | | 83 | 10368 |
| Dyrskjot_Bladder_3 | Dyrskjot L | 15173019 | GSE3167 | 60 | 22215 |
| Dyrskjot_Bladder_4 | Dyrksjot L | 12469123 | GSE88, GSE89, | 74 | 7129 |
| | | | GDS184, 3 | | |
| Dyrskjot_Bladder_5 | Dyrksjot L | 15930337 | | 29 | 59619 |
| Lindgren_Bladder | Lindgren D | 16532037 | | 75 | 7955 |
| Modlich_Bladder_2 | Modlich O | 15161696 | | 9 | 22215 |
| Modlich_Bladder_3 | Modlich O | 15161696 | | 54 | 1168 |
| Sanchez- | Sanchez- | 16432078 | | 157 | 22215 |
| Carbayo_Bladder_2 | Carbayo M | | | | |
| Stransky_Bladder | Stransky N | 17099711 | | 57 | 8555 |
| Multi-Cancer Studies | | | | | |
| Bittner_Multi-cancer | n/a | n/a | International Genomics | 14 | 54675 |
| | | | Consortium's expO | | |
| | | | data set GSE2109 | | |
| Ramaswamy_Multi- | Ramaswamy S | 11742071 | | 11 | 14018 |
| cancer | | | | | |
| Su_Multi-cancer | Su A I | 11606367 | | 8 | 10896 |

| | | |
|---|---|---|
| Total | 631 | 241298 |

T-tests conducted in Oncomine to assess for differential expression between classes were two-sided for differential expression analysis and one-sided for over-expression analysis. To account for multiple hypothesis testing, Q values (estimated false discovery rates) were calculated as the estimated number of false positives/number of called positives at a given p value (Storey et al. (2003) PNAS100:9440-9445).

Meta profiling is an automated process and proceeds along the following algorithm based upon the minimum meta-false discovery rate (mFDR_{MIN}) method as previously described (Rhodes et al. (2004) PNAS 101:9309-9314). Briefly, after choosing similar differential expression analyses, the direction and significance threshold are set to define the differential expression signatures from the pre-computed differential expression analyses, e.g. genes over-expressed in high grade versus low grade cancer at a threshold of 0.10 with a false discovery rate, Q, <0.10. For each clinical category, a meta-profile of significantly up- and down-regulated genes is created. The genes are then sorted based on the number of meta-profiles in which they are present and a meta-signature is defined if there are significantly more genes intersecting a given number of meta-profiles than would be expected by chance, as defined by a random simulation. Two meta-profiles were created for each of seven clinical categories: one list incorporated more studies and tolerated a less significant threshold and a second list allowed for differential expression in fewer studies while requiring a more significant differential expression (Table 2). Based on the idea of clinically aggressive or indolent bladder cancer, these lists were compared for overlap and pared into a single meta-signature of up- and down-regulated genes in aggressive disease. With the addition of housekeeping, outlier, and historic marker genes, this resulted in a meta-signature of 96 genes whose assays were pre-loaded onto the TLDA card (Table 3).

### Sample Preparation

### RNA Extraction

Frozen tissue blocks were retrieved from the University of Michigan bladder tumor bank, and had been previously pathologically reviewed to ensure adequate tissue and tumor representation. RNA isolation was performed using Trizol extraction (Invitrogen, Carlsbad, Calif.), and total RNA content was measured. Two micrograms of RNA was used for reverse transcription into cDNA, then purified with a Microcon YM-30 centrifugal filter device (Millipore Corp., Billerica, Mass.). This final cDNA was split into two aliquots of one microgram each; one aliquot was loaded onto the TLDA cards, and one stored at −80° C.

### QPCR Reproducibility

To assess reproducibility across TLDA card batches, one microgram of stored frozen cDNA from 16 samples run on the first three card sets (four samples from the first set, eight samples from the second set, and four samples from the third set) were re-run on cards from the fourth set.

### Benign

A hematoxylin and eosin (H&E) slide was generated from each frozen block for evaluation, and epithelium-rich areas were marked for microdissection. Microdissection of these areas was performed from the frozen block with a scalpel, using the noted area on the H&E slide as a guide. RNA extraction was performed with the Qiagen miRNeasy Mini Kit (Germantown, Md.). Two micrograms of RNA was processed further in an identical manner to the frozen bladder tumor specimens, and one microgram of cDNA from each benign specimen was loaded onto the TLDA cards.

### Molecular Concepts Map

The MCM consisted of various molecular concepts, or sets of biologically related genes, used for association analysis. Concepts consisted of three general types: 1) microarray-based gene expression profiles (from published data in the literature or from Oncomine analysis), 2) computed biological regulatory networks, and 3) gene and protein annotations from external databases. Microarray-based gene expression profiles derived from both normal and cancer tissue studies. Gene and protein information included chromosome location, biological processes, signaling and metabolic pathways, and protein families, interactions, and complexes. Computed networks were derived from transcription motifs and conserved promoter and 3′UTR elements (Tomlins et al. (2007) Nat. Genet. 39:41-51). A gene set was uploaded, and pairwise associations were generated between the gene set of interest and all selected concept types. Each association was assigned a p-value, with details of gene overlap between the sets, if applicable.

### Tissue Microarray and Immunohistochemistry

A bladder cancer progression tissue microarray was constructed from 41 cases representing benign bladder tissue, bladder CIS (carcinoma in situ), bladder cancer (non-invasive and invasive), and bladder cancer lymph node metastases. Three cores (0.6 mm in diameter) were taken from each representative tumor focus confirmed by two surgical pathologists. All tissues were derived from the University of Michigan bladder cancer database with informed consent of the patients and prior institutional review board approval.

Cases with staining in any cancerous epithelial cells were deemed positive. Overall ACTN1 and CDC25B expression was scored in a blinded fashion by two pathologists as negative (score=1), weak (score=2), moderate (score=3), or strong (score=4); scoring was based on the staining intensity and percentage of cells exhibiting that staining intensity, using a previously-validated system (4, 5). Product scores (intensity×percentage values) were calculated for all tissue cores, and the median product scores were determined for specific tissue types. These values were used in subsequent statistical analyses.

### Results and Discussion

### QPCR Reproducibility

Median correlation of the log-transformed gene expression values was 0.988 across 16 re-run samples (range 0.924-0.997), indicating high QPCR result reproducibility across batches (FIG. 5).

Univariate Associations between Clinical Variables and Progression

Of the clinical variables investigated (age, gender, pathologic stage, histological grade, and associated CIS), the only significant predictor of progression was pathologic stage (p=0.01; Table 5). Histological grade (high versus low grade) approached significance as a predictor of T2 progression (p=0.08) on univariate analysis; but when examined within patients of a single stage (Ta or T1), grade did not represent a significant predictor of progression [Ta (p=0.27), T1 (p=0.91)]. Indeed, the observed association between grade and progression is driven by the strong association between histological grade and pathologic stage in this cohort (Fisher exact test: p<0.0001).

TABLE 5
Univariate associations between clinical variables
and progression outcomes for 62 non-T2 patients

| | Univariate | |
|---|---|---|
| | Hazard Ratio | |
| Clinical Parameter | (95% CI) | p-value |
| Age | 2.0 | P = 0.15 |
| (>68.7 vs. <=68.7)^{#} | (0.8, 5.0) | |
| Gender | 1.8 | P = 0.34 |
| (Male vs. Female) | (0.5, 6.2) | |
| Pathologic Stage | 3.7 | P = 0.01* |
| (T1 vs. Ta) | (1.3, 10.1) | |
| Pathologic Grade | 2.3 | P = 0.08 |
| (HG vs. LG) | (0.9, 6.1) | |
| Pathologic Grade | 0.9 | P = 0.91 |
| Stage T1 Only | (0.3, 3.3) | |
| (HGT1 vs. LGT1) | | |
| Pathologic Grade | 3.0 | P = 0.27 |
| Stage Ta Only | (0.4, 21.6) | |
| (HGTa vs. LGTa) | | |
| Associated CIS | 1.6 | P = 0.34 |
| (Yes vs. No) | (0.6, 3.9) | |

^{#}Note:
68.7 years represents the median age for the non-T2 patients in this dataset.

### Prediction Quality as a Function of Gene Set Size

In developing the gene signature for prediction of progression to T2 disease, the use of different p-value cutoffs (p<0.01, p<0.005, and p<0.001) resulted in different numbers of genes that significantly differentiated between T2 and non-T2 patients. The predictive ability of each of these gene sets was investigated using the same procedure used for the 57-gene set (Table 6). While performance within the subset of stage Ta patients was relatively independent of gene set size, making effective predictions with a smaller number of genes is possible, as can be seen by examining the gene weights in Table 7; several genes contribute more strongly to the signature than others. However, because the original set of 90 profiled genes represented a meta-signature of aggressive behavior in bladder cancer across multiple microarray studies, many of the down-weighted genes from the 57-gene signature play an important role in prediction of progression to T2 disease.

TABLE 6
Varying Gene Signature Performance based
on P-Value Cutoff Used for Gene Selection

| | | p-value | p-value |
|---|---|---|---|
| | | (High Risk vs. | (High Risk vs. |
| p-value | Gene | Low Risk, T1 | Low Risk, Ta |
| Cutoff | Set Size | patients only)* | patients only)* |
| p < 0.05 | 57 | 0.02 | 0.03 |
| p < 0.01 | 45 | 0.31 | 0.02 |
| p < 0.005 | 39 | 0.05 | 0.02 |
| p < 0.001 | 27 | 0.13 | 0.02 |

*Reported p-values are from log-rank tests comparing patients categorized as high vs. low risk by each gene signature.

TABLE 7
List of 57 genes (37 up-regulated, 20 down-regulated) included in the
progression signature with full name, original meta-signature class,
univariate Wilcoxon rank-sum test p-values, and signature weights.

| | | | | |
|---|---|---|---|---|
| Gene | Full Name | Class | p-value | Weight |

Up-regulated

| | | | | |
|---|---|---|---|---|
| ACTN1 | Actinin, alpha 1 | Up | 0.011 | −0.076 |
| ADAM12 | ADAM metallopeptidase domain 12 | Up | <0.001 | 0.105 |
| APOC1 | apolipoprotein C-I | Up | <0.001 | 0.049 |
| BIRC5 | baculoviral IAP repeat- containing 5 | Historic | <0.001 | 0.068 |
| CALD1 | caldesmon 1 | Up | 0.012 | 0.006 |
| CALU | Calumenin | Up | <0.001 | 0.109 |
| CCL11 | chemokine (C-C motif) ligand 11 | Up | <0.001 | −0.009 |
| CCNB2 | cyclin B2 | Up | 0.02 | 0.062 |
| CDC25B | cell division cycle 25 homolog B | Up | <0.001 | 0.102 |
| CDC25C | cell division cycle 25 homolog C | Up | 0.05 | −0.044 |
| CDC6 | cell division cycle 6 homolog | Up | 0.01 | 0.029 |
| CDH11 | cadherin 11, type 2 | Up | <0.001 | 0.029 |
| CENPF | centromere protein F, 350/400ka | Up | 0.001 | 0.074 |
| COL6A3 | collagen, type VI, alpha 3 | Up | 0.001 | 0.069 |
| CSPG2 | Versican | Up | <0.001 | 0.095 |
| CXCL2 | chemokine (C-X-C motif) ligand 2 | Up | <0.001 | 0.007 |
| CYR61 | cysteine-rich, angiogenic inducer, 61 | Up | 0.012 | −0.089 |
| DOC1 | filamin A interacting protein 1-like | Up | <0.001 | 0.1 |
| EZH2 | enhancer of zeste homolog 2 | Historic | 0.009 | 0.051 |
| FAP | fibroblast activation protein, alpha | Up | <0.001 | 0.067 |
| FN1 | fibronectin 1 | Up | <0.001 | 0.075 |
| KIF2C | kinesin family member 2C | Up | 0.006 | 0.122 |
| MELK | maternal embryonic leucine zipper kinase | Up | 0.003 | <0.001 |
| MFAP2 | micro fibrillar-associated protein 2 | Up | <0.001 | −0.162 |
| MKI67 | antigen identified by monoclonal antibody Ki-67 | Historic | 0.001 | 0.035 |
| MMP11 | matrix metallopeptidase 11 | Up | <0.001 | 0.156 |
| MMP8 | matrix metallopeptidase 8 | Historic | 0.015 | 0.051 |
| MTHFD2 | methylenetetrahydrofolate dehydrogenase (NADP+ dependent) | Up | 0.005 | 0.111 |
| | 2, methenyltetrahydrofolate cyclohydrolase | | | |
| MYBL2 | v-myb myeloblastosis viral oncogene homolog (avian)-like 2 | Up | <0.001 | 0.067 |
| NNMT | nicotinamide N-methyltransferase | Up | <0.001 | 0.039 |
| POSTN | periostin, osteoblast specific | Up | <0.001 | 0.155 |
| SPARC | secreted protein, acidic, cysteine-rich | Up | 0.012 | 0.005 |
| THBS2 | thrombospondin 2 | Up | 0.001 | 0.063 |
| TIMP2 | TIMP metallopeptidase inhibitor 2 | Historic | 0.003 | 0.004 |
| TNC | tenascin C | Up | <0.001 | 0.027 |
| TNFAIP3 | tumor necrosis factor, alpha-induced protein 3 | Up | 0.006 | 0.037 |
| UBE2C | ubiquitin-conjugating enzyme E2C | Up | 0.002 | 0.065 |

Down-regulated

| | | | | |
|---|---|---|---|---|
| ANK1 | ankyrin 1, erythrocytic | Down | 0.034 | −0.322 |
| AQP3 | aquaporin 3 | Down | <0.001 | −0.096 |
| BMP7 | bone morphogenetic protein 7 | Down | 0.002 | −0.146 |
| CASP1 | caspase 1, apoptosis-related cysteine peptidase | Down | 0.003 | −0.1 |
| CD46 | CD46 molecule, complement regulatory protein | Down | 0.005 | 0.056 |
| CYP4F11 | cytochrome P450, family 4, subfamily F, polypeptide 11 | Down | 0.014 | −0.142 |
| DGKA | diacylglycerol kinase, alpha 80 kDa | Down | 0.022 | 0.019 |
| ERBB3 | v-erb-b2 erythroblastic leukemia viral oncogene homolog 3 | Historic | 0.003 | −0.046 |
| ERBB4 | v-erb-b2 erythroblastic leukemia viral oncogene homolog 4 | Historic | 0.01 | −0.189 |
| EVI1 | ecotropic viral integration site | Down | <0.001 | −0.275 |
| GPC3 | glypican 3 | Outlier | 0.003 | −0.102 |
| LGALS4 | lectin, galactose binding, soluble 4 | Outlier | 0.002 | −0.372 |
| MGST2 | microsomal glutathione S-transferase 2 | Down | 0.001 | −0.011 |
| MMP10 | matrix metallopeptidase 10 | Outlier | 0.033 | −0.143 |
| MMP16 | matrix metallopeptidase 16 | Up | 0.009 | −0.382 |
| RB1 | retinoblastoma 1 | Historic | <0.001 | −0.268 |
| SORL1 | sortilin-related receptor, L(DLR class) A repeats-containing | Down | 0.003 | 0.04 |
| ST3GAL5 | ST3 beta-galactoside alpha-2,3-sialyltransferase 5 | Down | <0.001 | −0.169 |
| TCEAL1 | transcription elongation factor A (SII)-like 1 | Down | 0.001 | −0.069 |
| TP53 | tumor protein p53 | Historic | <0.001 | −0.109 |

Weights are reported on a standardized scale, i.e., each gene has been scaled to have mean 0 and variance 1 prior to computing the weights; this makes it possible to assess the relative contribution of each gene to the overall signature.

### Development of the Gene Signature and Details of the Cross-Validation Procedure

Development of the 57-gene signature involved the following steps. First, each of the 90 non-housekeeping genes was evaluated in its ability to discriminate the 34 T2 patients from the 62 non-T2 patients by performing univariate Wilcoxon tests, and genes with p-values <0.05 were retained. Second, each gene's expression data [under the transformation: log(expression+1)] was truncated at the third standard deviation in the upward direction and this modified data was used as the set of variables in principal components analysis for all 96 patients. Third, the top 10 principal components, which explained 75% of the variance in the dataset, were used as predictors in a Cox regression model for these 96 patients, with T2 patients coded as having time-to-event=0 because they had experienced their outcome at time of TURBT; the best Cox model was selected via backwards selection using AIC to discriminate between models. Table 7 details each gene incorporated into the final signature, with its initial meta-signature class, p-value from univariate Wilcoxon rank-sum test, and ultimate algorithm weight in the signature. The gene weights were computed by decomposing the linear predictor obtained from the Cox model, via the principal component weights, into components for each of the 57 genes.

32/37 (86.5%) over-expressed genes were predicted to be over-expressed in aggressive cancers from meta-profiling and the remainder were historic markers; 12/20 (60%) under-expressed genes were predicted to be under-expressed from meta-profiling, with seven historic markers or outliers and one gene, matrix metalloprotenaise 16 (MMP16), initially predicted to be over-expressed.

Leave-one-out cross-validation was used to evaluate the predictive ability of this signature. Because 34 patients had already progressed to T2 prior to TURBT, the cross-validation procedure used these 34 patients only for training purposes. Thus, the cross-validation was performed as follows. Each of the 62 non-T2 patients was, in turn, removed from the dataset, and data from the remaining 61 non-T2 patients and the 34 T2 patients were used as a training set. Each stage of the above procedure—gene selection using Wilcoxon tests (cutoff: p <0.05), outlier truncation (above the third standard deviation for each gene), principal components analysis (retaining enough principal components to explain >75% of the variance in the data), and selection of Cox models (backwards selection using AIC)—was repeated for this training set, and the resulting Cox model was used to evaluate the predicted probability of T2 progression within two years for the left-out patient. In this way, estimates of each patient's probability of T2 progression were obtained using a model that was built without the use of that patient's expression data.

### MCM Analysis

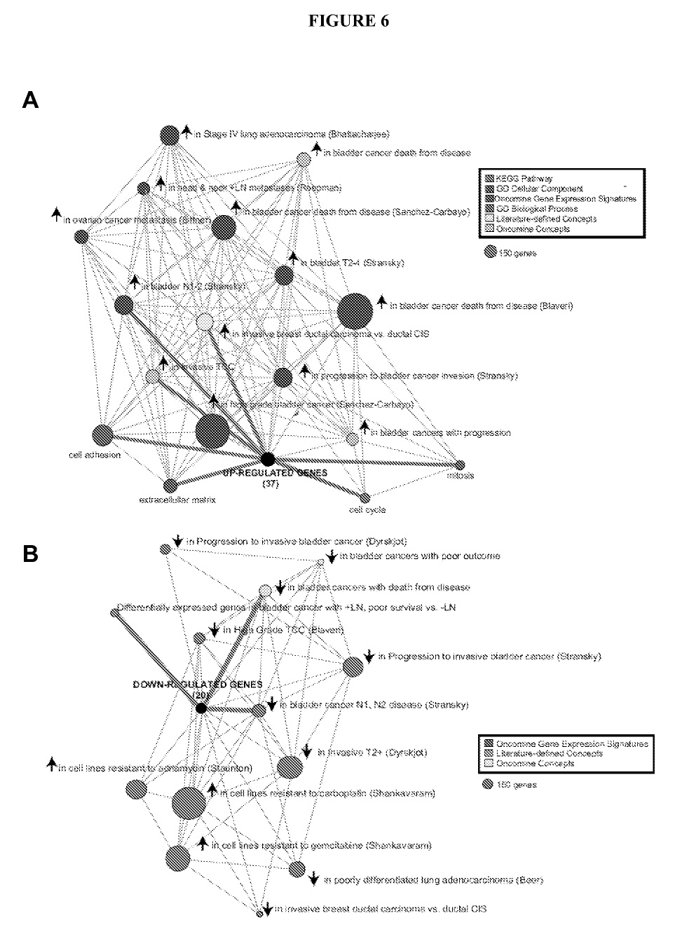

FIG. 6A demonstrates that the set of 37 over-expressed genes showed the most significant overlap with bladder cancer datasets versus other cancer types, indicating a mostly bladder cancer-specific transcriptional program. Furthermore, this over-expressed gene set overlapped with cell adhesion and extracellular matrix invasion pathways as well as cell cycle regulation and mitosis, confirming the importance of these programs in progression to muscle invasion in bladder cancer (Sanchez-Carbayo et al. (2002) Cancer Res. 62:6973-6980). Specifically, the first subset of 25 genes in the larger set of 37 over-expressed genes demonstrated greater enrichment with the extracellular matrix and cell adhesion concepts, while the second subset of 12 genes demonstrated significant overlap with the mitosis concept. Of other cancer types, genes that distinguish invasive breast ductal carcinoma from ductal carcinoma in-situ overlap with the set of 37 over-expressed genes as well as the cell adhesion concepts, implicating adhesion-related genes in both cancers. The over-expressed genes in T2 disease also demonstrated overlap with over-expressed genes in other advanced stage or metastatic cancers, including stage IV lung adenocarcinoma, head and neck squamous cell carcinoma nodal metastases, and metastatic ovarian carcinoma. While these overlaps were less significant than those with other bladder-related concepts, this supports the involvement of common genes in progression across various cancer types.

For the under-expressed set of 20 genes, these demonstrated again most significant overlap with bladder cancer datasets, and overlap with under-expressed genes in poorly differentiated lung adenocarcinoma (FIG. 6B). The under-expressed genes also demonstrated overlap with genes under-expressed in invasive breast ductal carcinoma versus ductal carcinoma in situ, providing further support for gene and pathway commonality between these two phenomena. Several concepts related to resistance of chemotherapy also arose, including those genes over-expressed in cell lines resistant to gemcitabine and adriamycin. These chemotherapeutic agents are used in adjuvant/neoadjuvant treatment of metastatic bladder cancer (with the MVAC (methotrexate/vinblastine/adriamycin/cisplatin) or gemcitabine/cisplatin standard-of-care protocols), and tumors with relative under-expression of these overlapping genes may possess a chemosensitivity to these agents. Finally, included in the list of down-regulated genes are several well-known tumor suppressors, including p53 and RB1 (retinoblastoma 1).

### Up-Regulated Gene Details

Up-regulated genes in this signature map showed the most significant overlap with bladder cancer datasets (p-value of enrichment with bladder cancer node status, 4.3×10⁻³³) versus other cancer types (p-value of highest enrichment with head and neck squamous cell carcinoma lymph node metastases, 7.4×10⁻¹⁶), implicating a bladder-specific program. Specifically, Cluster 1A of this gene set enriched for pathways of extracellular matrix cell adhesion (with an overlap of nine genes: CDH11, POSTN, COL6A3, FN1, TNC, CYR61, CCL11, THBS2, and ADAM12), while Cluster 1B demonstrated more overlap with cell cycle and mitosis pathways (five genes: KIF2C, UBE2C, CCNB2, CDC6, and CDC25B).

In terms of this gene set overlapping with the literature-define concept of genes up-regulated in invasive ductal carcinoma versus ductal CIS, the association had a p-value of 2.7×10⁻¹⁶ and overlap of 12 genes (CDH11, POSTN, COL6A3, CSPG2, FAP, FN1, CYR61, MFAP2, MMP11, NNMT, SPARC, THB S2, CALD1, and ADAM12).

### Down-Regulated Gene Details

Down-regulated genes again demonstrate the most significant overlap with bladder cancer datasets (p-value of enrichment with bladder cancer N1-2 disease, 1.3×10⁻¹²) versus other cancer types (p-value of enrichment with poorly differentiated lung adenocarcinoma, 5.8×10⁻⁴). The overlap with genes down-regulated in invasive ductal carcinoma versus ductal CIS consisted of three genes (ERBB3, ERBB4, and MMP10) and had a p-value of 5×10⁻⁵.

### Immunohistochemistry

Immunohistochemistry was performed to investigate ACTN1 and CDC25B protein expression on bladder cancer progression tissue microarray. Again, ACTN1 showed a significant differential expression between normal bladder tissues and non-invasive tumors versus invasive and metastatic tumors (Scheffe: p=0.002), with an estimated mean difference of 129 product score units (95% CI: 46, 212). The most significant individual group comparison difference was between the non-invasive and either invasive or metastatic groups (Tukey's HSD: p=0.003 for each). On the same progression tissue microarray, CDC25B demonstrated a more gradually increasing trend from normal to non-invasive to invasive to metastatic bladder cancer; pairwise between-group comparisons revealed higher CDC25B expression in the more severe diagnosis group without exception. Therefore, CDC25B expression was assumed to exhibit a linear trend by diagnosis group, resulting in a simpler model (F-test for linear trend vs. general ANOVA model: p=0.97). CDC25B mean product scores increased by 56 product score units (95% CI: 28,83) between each diagnosis group. This increase was highly significant (p=0.0002), confirming the gradual upward trend of CDC25B expression with disease severity.

### Immunohistochemistry Candidates

ACTN1 is a member of the spectrin gene superfamily of cytoskeletal proteins, and is involved in cross-linking actin in various cell types (Lazarides et al. (1975) Cell 6:289-298). It is also thought to be an important upstream component of the mechanical response pathway mediating pressure-stimulated cell adhesion in colon cancer (Craig et al. (2008) Neoplasia 10:217-222), and in colon, bladder, and prostate cancers, has been identified as possessing a tumor-specific splice variant (Thorsen et al. (2008) Proteomics 7:1214-1224). In bladder cancer, the change in splicing pattern is more pronounced in T2 versus Ta tumors, demonstrating an ability to utilize ACTN1 in stage separation. In experiments conducted during the course of the development of the present invention, ACTN1 protein expression was also significantly different between non-muscle-invasive and invasive or metastatic bladder cancers.

CDC25B is a member of the CDC25 phosphatase family of proteins, which activate cyclin-dependent kinases to regulate progression through the cell cycle. They are also important in the checkpoint pathways activated in response to DNA stress to prevent further cell division and growth (Boutros et al. (2007) Nat. Rev. Cancer 7:495-507). CDC25B protein overexpression, in particular, has been associated with several cancer types, including colorectal (Takemasa et al. (2000) Cancer Res. 60:3043-3050), gastric (Kudo et al. (1997) Jpn. J. Cancer Res. 88:947-952), endometrial (Tsuda et al. (2003) Oncology 65:159-166), and prostate cancer (Ngan et al. (2003) Oncogene 22:734-439). However, no definitive studies have been published on CDC25B in bladder cancer explicitly, other than its initial inclusion in the top 200 gene markers distinguishing progressive non-muscle-invasive bladder cancers (Dyrskjot et al. (2005) Clin. Cancer Res. 11:4029-4036). In that microarray study, CDC25B gene expression is up-regulated in tumors which progress, correlating with the gradually up-regulated protein expression on immunohistochemistry seen in the present study.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method for diagnosing bladder cancer, said method comprising:
a) quantifying the level of expression of at least one gene in a bladder tissue sample from a subject, said at least one gene selected from the group consisting of ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C—X—C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53); and
b) determining the presence of at least one bladder tumor based on said level of expression of said at least one gene.

2. The method of claim 1, wherein over-expression of at least one gene selected from the group consisting of ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), and UBE2C (ubiquitin-conjugating enzyme E2C) is indicative of presence of said bladder tumor.

3. The method of claim 1, wherein under-expression of at least one gene selected from the group consisting of ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53) is indicative of presence of said bladder tumor.

4. The method of claim 1, wherein said bladder tissue sample comprises a tumor biopsy.

5. The method of claim 1, further comprising the step of comparing said gene expression levels to the expression level of said genes in a second sample.

6. The method of claim 5, wherein the second sample comprises a benign bladder tissue sample from said subject.

7. The method of claim 5, wherein the second sample comprises a historical bladder tissue sample from said subject.

8. The method of claim 1, wherein said method is executed more than once at periodic intervals.

9. The method of claim 1, further comprising the step of determining the stage of at least one bladder tumor based on said level of expression of said at least one gene.

10. A method of analyzing a bladder tumor, said method comprising
a) quantifying the level of expression of at least one gene in a bladder tissue sample from a subject, said at least one gene selected from the group consisting of ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53); and
b) determining risk of said bladder tumor to invade bladder muscular tissue of a subject based on said level of expression of said at least one gene.

11. The method of claim 10, wherein over-expression of at least one gene selected from the group consisting of ACTN1 (actinin, alpha 1), ADAM12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C-X-C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), and UBE2C (ubiquitin-conjugating enzyme E2C) is indicative of increased risk of said bladder tumor invading muscular tissue of the bladder.

12. The method of claim 10, wherein under-expression of at least one gene selected from the group consisting of ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53) is indicative of increased risk of said bladder tumor invading muscular tissue of the bladder.

13. The method of claim 10, wherein said bladder tissue sample comprises a tumor biopsy.

14. The method of claim 13, wherein the stage of said tumor has been determined.

15. The method of claim 13, wherein the stage of said tumor has not been determined.

16. The method of claim 14, wherein the stage of said tumor has been determined to be stage Ta, T is, or T1.

17. The method of claim 14, wherein the stage of said tumor has been determined to be stage T2 or higher.

18. The method of claim 10, further comprising the step of comparing said gene expression levels to the expression level(s) of said gene(s) in a second sample.

19. The method of claim 18, wherein the second sample comprises a benign bladder tissue sample from said subject.

20. The method of claim 18, wherein the second sample comprises a historical bladder tissue sample from said subject.

21. The method of claim 10, wherein said method is executed more than once at periodic intervals.

21. A kit comprising reagents configured to quantify the expression of one or more genes selected from the group consisting of ACTN1 (actinin, alpha 1), ADAM 12 (ADAM metallopeptidase domain 12), APOC1 (apolipoprotein C-I), BIRC5 (baculoviral IAP repeat-containing 5), CALD1 (caldesmon 1), CALU (calumenin), CCL11 (chemokine (C-C motif) ligand 11), CCNB2 (cyclin B2), CDC25B (cell division cycle 25 homolog B), CDC25C (cell division cycle 25 homolog C), CDC6 (cell division cycle 6 homolog), CDH11 (cadherin 11, type 2), CENPF (centromere protein F, 350/400 ka), COL6A3 (collagen, type VI, alpha 3), CSPG2 (versican), CXCL2 (chemokine (C—X—C motif) ligand 2), CYR61 (cysteine-rich, angiogenic inducer, 61), DOC1 (filamin A interacting protein 1-like), EZH2 (enhancer of zeste homolog 2), FAP (fibroblast activation protein, alpha), FN1 (fibronectin 1), KIF2C (kinesin family member 2C), MELK (maternal embryonic leucine zipper kinase), MFAP2 (microfibrillar-associated protein 2), MKI67 (antigen identified by monoclonal antibody Ki-67), MMP11 (matrix metallopeptidase 11), MMP8 (matrix metallopeptidase 8), MTHFD2 (methylenetetrahydrofolate dehydrogenase (NADP+ dependent) 2, methenyltetrahydrofolate cyclohydrolase), MYBL2 (v-myb myeloblastosis viral oncogene homolog (avian)-like 2), NNMT (nicotinamide N-methyltransferase), POSTN (periostin, osteoblast specific), SPARC (secreted protein, acidic, cysteine-rich), THBS2 (thrombospondin 2), TIMP2 (TIMP metallopeptidase inhibitor 2), TNC (tenascin C), TNFAIP3 (tumor necrosis factor, alpha-induced protein 3), UBE2C (ubiquitin-conjugating enzyme E2C), ANK1 (ankyrin 1, erythrocytic), AQP3 (aquaporin 3), BMP7 (bone morphogenetic protein 7), CASP1 (caspase 1, apoptosis-related cysteine peptidase), CD46 (CD46 molecule, complement regulatory protein), CYP4F11 (cytochrome P450, family 4, subfamily F, polypeptide 11), DGKA (diacylglycerol kinase, alpha 80 kDa), ERBB3 (v-erb-b2 erythroblastic leukemia viral oncogene homolog 3), ERBB4 (v-erb-b2 erythroblastic leukemia viral oncogene homolog 4), EVI1 (ecotropic viral integration site), GPC3 (glypican 3), LGALS4 (lectin, galactose binding, soluble 4), MGST2 (microsomal glutathione S-transferase 2), MMP10 (matrix metallopeptidase 10), MMP16 (matrix metallopeptidase 16), RB1 (retinoblastoma 1), SORL1 (sortilin-related receptor, L(DLR class) A repeats-containing), ST3GAL5 (ST3 beta-galactoside alpha-2,3-sialyltransferase 5), TCEAL1 (transcription elongation factor A (SII)-like 1), and TP53 (tumor protein p53).

22. The kit of claim 21, wherein the kit comprises a QPCR card.

23. A method for diagnosing bladder cancer, said method comprising:
a) quantifying the level of expression of at least one gene in a bladder tissue sample from a subject, said at least one gene selected from the group consisting of ACTN1 (actinin, alpha 1) and CDC25C (cell division cycle 25 homolog C); and
b) determining the presence of at least one bladder tumor based on said level of expression of said at least one gene.

24. A method analyzing a bladder tumor, said method comprising:
a) quantifying the level of expression of at least one gene in a bladder tissue sample from a subject, said at least one gene selected from the group consisting of ACTN1 (actinin, alpha 1) and CDC25C (cell division cycle 25 homolog C); and
b) determining risk of said bladder tumor to invade bladder muscular tissue of a subject based on said level of expression of said at least one gene.
